SEQUENCE LISTING (28 sequences)

SEQ ID NO: 1 - protein, 7 aa, Artificial Sequence
  Synthetic Construct
KIWKLPQ

SEQ ID NO: 2 - protein, 7 aa, Artificial Sequence
  Synthetic Construct
KVWSLPQ

SEQ ID NO: 3 - protein, 7 aa, Artificial Sequence
  Synthetic Construct
KLWVLPK

SEQ ID NO: 4 - protein, 7 aa, Artificial Sequence
  Synthetic Construct
KIFVWPY

SEQ ID NO: 5 - protein, 7 aa, Artificial Sequence
  Synthetic Construct
KIWVLPQ

SEQ ID NO: 6 - protein, 7 aa, Artificial Sequence
  Synthetic Construct
KXWXLPX

SEQ ID NO: 7 - protein, 7 aa, Artificial Sequence
  Synthetic Construct
STFGLQD

SEQ ID NO: 8 - protein, 7 aa, Artificial Sequence
  Synthetic Construct
IGPATRS

SEQ ID NO: 9 - protein, 7 aa, Artificial Sequence
  Synthetic Construct
LPKELRH

SEQ ID NO: 10 - protein, 7 aa, Artificial Sequence
  Synthetic Construct
INYHVQK

SEQ ID NO: 11 - protein, 7 aa, Artificial Sequence
  Synthetic Construct
GPAVLTL

SEQ ID NO: 12 - protein, 7 aa, Artificial Sequence
  Synthetic Construct
TPLPANL

SEQ ID NO: 13 - protein, 7 aa, Artificial Sequence
  Synthetic Construct
HRSPHTP

SEQ ID NO: 14 - protein, 7 aa, Artificial Sequence
  Synthetic Construct
YNLPPST

SEQ ID NO: 15 - protein, 7 aa, Artificial Sequence
  Synthetic Construct
YHRPPPY

SEQ ID NO: 16 - protein, 7 aa, Artificial Sequence
  Synthetic Construct
SAPPSHP

SEQ ID NO: 17 - protein, 7 aa, Artificial Sequence
  Synthetic Construct
KGEHPTV

SEQ ID NO: 18 - protein, 7 aa, Artificial Sequence
  Synthetic Construct
GANMPHP

SEQ ID NO: 19 - protein, 7 aa, Artificial Sequence
  Synthetic Construct
NHHPLPR

SEQ ID NO: 20 - protein, 7 aa, Artificial Sequence
  Synthetic Construct
SLSPNLS

SEQ ID NO: 21 - protein, 7 aa, Artificial Sequence
  Synthetic Construct
GKIWTTP

SEQ ID NO: 22 - protein, 7 aa, Artificial Sequence
  Synthetic Construct
WPLGASR

SEQ ID NO: 23 - protein, 7 aa, Artificial Sequence
  Synthetic Construct
HWLPLYG

SEQ ID NO: 24 - protein, 7 aa, Artificial Sequence
  Synthetic Construct
HWGSLRA

SEQ ID NO: 25 - protein, 7 aa, Artificial Sequence
  Synthetic Construct
HYLPLYW

SEQ ID NO: 26 - protein, 7 aa, Artificial Sequence
  Synthetic Construct
PWKKLLV

SEQ ID NO: 27 - protein, 11 aa, Artificial Sequence
  Synthetic Construct
KLWVLPKGGGC

SEQ ID NO: 28 - protein, 4 aa, Artificial Sequence
  Synthetic Construct
GGGC

(19) United States
(12) Patent Application Publication
(10) Pub. No.: US 2011/0143993 A1
(43) Pub. Date: Jun. 16, 2011

(54) ENDOTHELIAL BASEMENT MEMBRANE TARGETING PEPTIDE LIGANDS

(75) Inventors: Robert S. Langer, (US); Omid C. Farokhzad, Chestnut Hill, MA (US); Juliana M. Chan, Cambridge, MA (US)

(73) Assignees: The Brigham and Women's Hospital, Inc.; Massachusetts Institutes of Technology

(21) Appl. No.: 12/969,345

(22) Filed: Dec. 15, 2010

Related U.S. Application Data
(60) Provisional application No. 61/286,650, filed on Dec. 15, 2009.

(51) Int. Cl. A61K 38/08; C07K 7/06; A61P 35/00; A61P 27/02; A61P 9/10; A61P 7/00; G01N 33/68

(52) U.S. Cl. 514/1.4; 530/329; 514/21.7; 514/19.2; 514/13.3; 514/17.2; 514/21.6; 530/327; 435/7.8

(57) ABSTRACT

Peptides that selectively bind to antigens exposed in vascular disease or dysfunction have been identified by biopanning a phage library. The ligands are useful when attached to a substrate to be in contact with endothelial surfaces, especially those where drug delivery is utilized, such as following angioplasty, with release from a drug delivery reservoir in a medical device such as a stent, or by administration intravenously in the form of nano or microparticles, although the peptides may also be used with other medical devices or substrates, for targeting or to increase adhesion to endothelial surfaces. The nanoparticle technology can be used to treat injured vasculature, a clinical problem of primary importance. The targeted nanoparticles are also useful in the treatment of other diseases and disorders such as oncologic and regenerative diseases and indications where neoangiogenesis is commonly observed.

### CROSS REFERENCE TO RELATED APPLICATIONS

This application claims benefit of U.S. Provisional Application No. 61/286,650, filed Dec. 15, 2009, which is hereby incorporated herein by reference in its entirety.

### STATEMENT REGARDING FEDERALLY SPONSORED RESEARCH OR DEVELOPMENT

This invention was made with Government Support under Agreement CA 119349 and EB003647 awarded to Robert S. Langer by the National Institutes of Health and National Cancer Institute. The Government has certain rights in the invention.

### FIELD OF THE INVENTION

The present invention is generally in the field of endothelial basement membrane targeting peptide ligands, and methods of manufacture and use, particularly in the area of nanoparticles.

### BACKGROUND OF THE INVENTION

Tight junctions, or zonula occludins, are the closely associated areas of two cells whose membranes join together forming a virtually impermeable barrier to fluid. It is a type of junctional complex present only in vertebrates. The corresponding junctions that occur in invertebrates are septate junctions. Tight junctions are composed of a branching network of sealing strands, each strand acting independently from the others. Therefore, the efficiency of the junction in preventing ion passage increases exponentially with the number of strands. Each strand is formed from a row of transmembrane proteins embedded in both plasma membranes, with extracellular domains joining one another directly. Although more proteins are present, the major types are the claudins and the occludins. These associate with different peripheral membrane proteins located on the intracellular side of plasma membrane, which anchor the strands to the actin cytoskeleton. Thus, tight junctions join together the cytoskeletons of adjacent cells.

The tight junctions perform three vital functions. They hold cells together. They help to maintain the polarity of cells by preventing the lateral diffusion of integral membrane proteins between the apical and lateral/basal surfaces, allowing the specialized functions of each surface, for example, receptor-mediated endocytosis at the apical surface and exocytosis at the basolateral surface, to be preserved. This preserves the transcellular transport. They prevent the passage of molecules and ions through the space between cells. So materials must actually enter the cells (by diffusion or active transport) in order to pass through the tissue. This pathway provides control over what substances are allowed through. Tight junctions play this role in maintaining the blood-brain barrier.

The endothelium is defined as a thin layer of flat epithelial cells that lines serous cavities, lymph vessels, and blood vessels. The term epithelia can refer to any tissue which is flattened (and possibly stratified). By this definition, the endothelium is a type of epithelium. However, there are a number of differences that exist. Epithelia line the “outside” of our bodies, such as the skin, intestines, bladder, urethra etc. while endothelia line the “inside” of our bodies, such as lymph and blood vessels. Endothelial cells have a different embryological derivation (mesodermal) from true epithelial cells (ectodermal and endodermal). Endothelial cells contain vimentin filaments while epithelial cells have keratin filaments. Generally, both the epithelium and endothelium comprise the outermost layer or lining of anatomical structures.

Epithelia are classed as ‘tight’ or ‘leaky’ depending on the ability of the tight junctions to prevent water and solute movement. Tight epithelia have tight junctions that prevent most movement between cells. An example of a tight epithelium is the distal convoluted tubule, part of the nephron in the kidney. Leaky epithelia do not have these tight junctions, or have less complex tight junctions. For instance, the tight junction in the kidney proximal tubule, a very leaky epithelium, has only two to three junctional strands, and these strands exhibit infrequent large slit breaks.

Many diseases and disorders are characterized by the presence of leaky junctions, particularly of the endothelial surface. For example, angioplasty, which removes a portion of the endothelial surface and sub-basement membrane and evokes inflammation, can cause leaky junctions which are associated with exposure of the basement membrane. This is also characteristic of many types of cancers and certain diseases such as sepsis and in premature babies.

It is an object of the present invention to provide proteins or peptides which selectively bind to epitopes exposed as a consequence of increased vascular permeability.

It is a further object of the present invention to provide particles or conjugates targeted to epitopes exposed as a consequence of increased vascular permeability, especially for targeted delivery of therapeutic, prophylactic or diagnostic agents, or mechanical barrier molecules which can be used to treat or diagnose these areas of increased vascular permeability.

It is still another object of the present invention to provide ligands that can specifically target disrupted basement membrane.

### SUMMARY OF THE INVENTION

Peptides that selectively bind to antigens exposed in vascular disease or dysfunction are used to target therapeutic, nutritional, diagnostic, prophylactic or barrier agents (referred to herein as “pharmaceutical agents”) to sites of disease or dysfunction such as the acute leaky junctions in sepsis, and the chronic changes in vascular permeability associated with restenosis, transplantation and in the gastrointestinal and pulmonary tracts of premature infants.

In one embodiment, the targeting ligands are identified by biopanning a phage library. As demonstrated by the examples, a number of heptapeptide ligands were identified by biopanning a phage library against collagen IV, which represents 50% of the vascular basement membrane, and then identifying specific ligands for targeting affinity against a Matrigel extract rich in collagen IV and laminin.

The ligands can be attached or conjugated to drugs, particles or polymers having a barrier function, polymers which are adhesive, polymers which are anti-adhesive, or a substrate to be in contact with endothelial surfaces, for example, a stent or catheter. These may be used following angioplasty, to provide release from a drug delivery reservoir in the stent, or used with other medical devices or substrates for targeting or to increase adhesion to disrupted endothelial surfaces. Alternatively, the conjugate can be administered intravenously in the form of nano or microparticles or as a conjugate of the targeting ligand attached directly to the pharmaceutical agents. The pharmaceutical agent to be delivered may be a pharmacologically active agent such as an anti-inflammatory, antibiotic, or anti-angiogenic, or it may be a physical barrier to leakage through the dysfunctional endothelium or a material which promotes, or prevents, adhesion to the endothelium.

As demonstrated by the examples, temporal control was achieved using 60-nm hybrid nanoparticles with a lipid shell interface surrounding a polymer core. The core was loaded with slow-eluting conjugates of paclitaxel, made by a modified ring-opening strategy, for controlled ester hydrolysis and drug release over approximately 12 days. The animal studies showed that the combination of these materials inhibited human aortic smooth muscle cell proliferation in vitro and showed greater in vivo vascular retention during percutaneous angioplasty over non-targeted controls. These studies established that the technology can be used to treat injured vasculature, a clinical problem of primary importance. When these vehicles were administered intraarterially (IA) or intravenously (IV), they demonstrated specific localization to injured vasculature and exhibited controlled drug release over approximately 10-12 days. The targeted nanoparticles are also useful in the treatment of other diseases and disorders such as oncologic and regenerative diseases and indications where neoangiogenesis is commonly observed.

### BRIEF DESCRIPTION OF THE DRAWINGS

FIG. 1A is a table of peptide sequences and graph of binding absorbance. Identification and characterization of peptides for targeting to injured vasculature. 23 phage clones from Rounds 3-5 of the phage display screen. Group A: Peptide sequences which show homology to resident basement membrane proteins or contain collagen binding-motifs analyzed by pBLAST against the NCBI homo sapiens non-redundant protein sequence database. Group B: Sequences resembling the collagen IV Gly-Pro-Pro (GPP) triple helix. Group C: Sequences with no identifiable relationship to resident basement membrane structures. The clones were tested against the random library (R0) for binding to Matrigel (lighter shaded bars) or bovine serum albumin (BSA) (black bars). Bound phages were labeled with peroxidase-conjugated anti-M 13 monoclonal antibodies (mAbs), and ABTS absorbance at 405 nm was read against a reference wavelength of 490 nm (mean±s.d., n=3). (**) P<0.01; (***) P<0.001, all compared with R0 (one-way analysis of variance with Tukey post-hoc test). FIG. 1B is a graph of the sequence-specific competition binding assays of phage clones A-8, A-9, C-10 and C-11 against synthetic peptide equivalents to Matrigel, plotted as normalized absorbance (%) versus peptide (M). IC₅₀ values were determined (SI Methods) and normalized on a percentage scale (mean±s.d., n=3). (▴) C-11; (▪) A-9; () C-10; (▾) A-8. FIG. 1C is a graph of the titer count analyses of C-11 compared to R0 on Matrigel and collagen IV. Titers of eluted phages were averaged to give the p.f.u./mL (mean±s.d., n=3). (***) P<0.001 by a paired two-sample Student's t-test.

FIG. 2A is a schematic of paclitaxel-polylactic acid (Ptxl-PLA) biomaterial synthesis. Ptxl was mixed with equimolar amounts of [(BDI)ZnN(TMS)₂]; the (BDI)Zn-Ptxl complex formed in situ initiated and completed the polymerization of lactide. For the nanoparticle core, Ptxl-PLA₂₅ drug conjugates which have approximately 25 dl-lactide monomer units were synthesized. FIG. 2B is a schematic of nanoparticle synthesis by nanoprecipitation and self-assembly. FIG. 2C is a graph of in vitro drug release of Ptxl from the nanoparticle core, plotted as percent drug retained versus time in days. Samples at different time points were measured for absorbance at 227 nm (mean±s.d., n=3).

FIG. 3 is a graph showing human aortic smooth muscle cell (haSMC) cytotoxicity studies as a function of binding affinity. HaSMC on Matrigel-coated plates were incubated with nanoparticles (T); scrambled-NPs (S); or non-targeted bare-NPs (B); four-fold dilutions of Ptxl without nanoparticles; and a media-only control for 45 min. Formazan product formation was measured at 490 nm against 650 nm reference wavelength (mean±s.d., n=5). (***) P<0.001 by one-way analysis of variance with Tukey post-hoc test.

FIG. 4 is a graph of the quantification of nanoparticle binding ex vivo to angioplastied aortas. Aorta sections (n=3) were analyzed using the region-of-interest (ROI) function of the IVIS Living Image Software and shown as average efficiency values per unit area (cm⁻²) of mean±s.d. (*) P<0.05, (**) P<0.01 by one-way analysis of variance with Tukey post-hoc test.

FIG. 5 is a graph of the quantification of nanoparticle binding in vivo to angioplastied left common carotids by intraarterial delivery. Both the left and right common carotid arteries (n=3) were analyzed using the region-of-interest (ROI) function of the IVIS Living Image Software and shown here as average efficiency per unit area (cm²) of mean±s.d. (*) P<0.05 by one-way analysis of variance with Tukey post-hoc test.

FIG. 6 is a graph of the quantification of nanoparticle binding in vivo to angioplastied left common carotids by intravenous delivery. Both the left and right common carotid arteries (n=5) were analyzed using the region-of-interest (ROI) function of the IVIS Living Image Software and shown here as average efficiency per unit area (cm²) of mean±s.d. (**) P<0.01, (***) P<0.001 by one-way analysis of variance with Tukey post-hoc test.

FIG. 7A is a schematic of targeted lipid-polymeric NP design. The targeted lipid-polymeric nanoparticles have a core-shell structure: soybean lecithin and peptide-conjugated distearoylphosphatidylethanolamine-poly(ethylene glycol) (DSPE-PEG-Peptide) forms the shell; poly(lactic-co-glycolic acid) (PLGA) encapsulating paclitaxel forms the core.

FIG. 7B is a dynamic light scattering plot showing the size ranges of targeted lipid-polymeric nanoparticles. Inset: Transmission electron micrograph (TEM) image of targeted lipid-polymeric nanoparticles. Scale, 100 nm.

FIG. 7C is a graph showing in vitro drug release (%) as a function of time (h) for paclitaxel (triangle), NP (circle), and targeted lipid-polymeric nanoparticle (diamond) formulations. Percentage drug release from samples placed in a PBS buffer sink at 37° C. with stirring.

FIG. 7D is a graph showing percentage of remaining ¹⁴C paclitaxel quantified as disintegrations per minute (DPM) in (▾) paclitaxel, (Δ) NP, and (▪) targeted lipid-polymeric nanoparticle samples in plasma. The graph also shows percentage of remaining ³H-PLGA in (∘) targeted lipid-polymeric nanoparticle samples in plasma. Inset (taken from shaded area on main graph): Percentage of remaining ¹⁴C-paclitaxel in plasma of paclitaxel, NP, and targeted lipid-polymeric nanoparticle samples on a log-scale Y-axis. All results are taken as mean±SEM, n=6.

FIG. 8 is a graph showing Neointima/Media (N/M) ratio measurements taken from the site of greatest luminal narrowing for each balloon-injured carotid artery without treatment (first bar), treated with paclitaxel (second bar=0.3 mg/kg, third bar=1 mg/kg), NP (fourth bar=0.3 mg/kg, fifth bar=1 mg/kg), or targeted lipid-polymeric nanoparticles (sixth bar=0.3 mg/kg, seventh bar=1 mg/kg). Animals were dosed on Day 0 and Day 5 post-surgery and the study was concluded on Day 14. All results are taken as mean±SEM, n=5. *, P<0.05; †, P<0.01 by one-way ANOVA with Tukey post-hoc test.

FIG. 9A is a schematic of phage display selection strategy. During the initial selection, the M13 bacteriophage library was panned against human collagen IV. In round 2 to round 5, the collagen IV enriched phage pool was in addition subtractively panned against human collagen I. 15 clones per round were randomly picked for further biochemical analysis and DNA sequencing.

FIG. 9B depicts amino acid sequence of the top four binding clones aligned by the CLUSTAL 2.0.10 multiple sequence alignment software to give a consensus sequence.

FIG. 9C is a graph showing normalized absorbance signal in sequence-specific competition binding assays of phage clones Seq-1 (), Seq-2 (▾), Seq-3 (▴), and Seq-4 (▪) against synthetic peptide equivalents ([peptide] M) to Matrigel. IC₅₀ values were determined and normalized on a percentage scale (mean±SD, n=3).

FIG. 9D is a bar graph showing absorbance of the chromogenic substrate (ABTS) (405-490 nm) for Seq-3 (open bars) and the library (R0, solid bars) for binding to Matrigel (first two bars), collagen IV (CIV, third and fourth bars), collagen I (CI, fifth and sixth bars), and bovine serum albumin (BSA, seventh and eighth bars). Bound phages were labeled with peroxidase-conjugated anti-M13 phage monoclonal antibodies, and absorbance of the chromogenic substrate (ABTS) was read at 405 nm against a reference wavelength of 490 nm.

FIG. 9E is a bar graph showing phage titer counts (pfu/mL) of Seq-3 (open bar) versus the library (R0, solid bar) on Matrigel (first two bars) and collagen IV (third and fourth bars). Titers of eluted phages were averaged to give values of pfu/mL. All results were taken as mean±SD, n=3. ***, P<0.001 by a paired two-sample Student's t-test.

FIG. 10 is a bar graph showing biodistribution (counts per tissue weight (DPM/g)) of ¹⁴C-paclitaxel encapsulated targeted lipid-polymeric nanoparticles in the liver (first bar), spleen (second bar), kidney (third bar), lung (fourth bar), heart (fifth bar) and blood (sixth bar) 24 h after intravenous injection. Radioactivity in tissue and blood samples are quantified as DPM per gram of tissue; mean±SD, n=6.

### DETAILED DESCRIPTION OF THE INVENTION

Conventional molecular targeting of relevant cell-based targets can be confounded by inter- and intra-patient heterogeneity in cell surface antigen expression, as described by Rajan, et al. (2009) Nat Rev Urol 6, 454-460 and Andrechek, et al. (2009) Proc Natl Acad Sci USA 106, 16387-16392. More recently, investigators have explored abundant non-cellular targets such as the coagulation cascade (Peters, et al. (2009) Proc Nall Acad Sci USA 106, 9815-9819), intra-articular cartilage (Rothenfluh, D. A., Bermudez, H., O'Neil, C. P., & Hubbell, J. A. (2008) Nat Mater 7, 248-254) and extracellular matrix (O'Neil, et al. (2009) J Control Release 137, 146-151).

Many human diseases are associated with compromised vasculature (Folkman, J. (2007) Nat Rev Drug Discov 6, 273-286; Ross, R. (1999) N Engl J Med 340, 115-126). These breaches could be targeted by targeting non-cellular protein epitopes which become abundantly available as a consequence of vascular permeability.

### I. Compositions

A. Endothelial Basement Membrane Targeting Peptide Ligands

The basement membrane is the fusion of two basal laminae. It consists of an electron-dense membrane called the lamina densa, about 30-70 nanometers in thickness, and an underlying network of reticular collagen (type III) fibrils (its precursor is fibroblasts) which average 30 nanometers in diameter and 0.1-2 micrometers in thickness. This type III collagen is of the reticular type, in contrast to the fibrillar collagen found in the interstitial matrix. In addition to collagen, this supportive matrix contains intrinsic macromolecular components. The Lamina Densa (which is made up of type IV collagen fibers; perlecan (a heparan sulfate proteoglycan) coats these fibers and they are high in heparan sulfate) and the Lamina Lucida (made up of laminin, integrins, entactins, and dystroglycans) together make up the basal lamina. Lamina Reticularis attached to basal lamina with anchoring fibrils (type VII collagen fibers) and microfibrils (fibrilin) is collectively known as the basement membrane.

In the preferred embodiment, the ligands bind to collagen I, II, III or IV, laminin, an integrin, an entactin or a dystroglycan. Collagen IV is 50% by mass of the basement membrane. Laminin is 30% of the basement membrane. The rest, heparan sulfate proteoglycans, all the glycans, perlecan, nidogen, etc, form the remaining 10-20%.

Peptides which bind specifically to epitopes exposed following vacular breach or other injury can be identified by screening of peptide libraries. This is demonstrated by the examples where peptides which bind specifically to antigen exposed following vascular breach or other injury were identified by screening of a peptide library for:

Peptide sequences which show homology to resident basement membrane proteins or contain collagen binding-motifs analyzed by pBLAST against the NCBI homo sapiens non-redundant protein sequence database.

Sequences resembling the collagen IV Gly-Pro-Pro (GPP) triple helix.

Sequences with no identifiable relationship to resident basement membrane structures. The clones were tested against the random library (R0) for binding to Matrigel (other extracellular matrix (ECM) material could be used) or bovine serum albumin (BSA) (or other non-ECM material). The alignment and consensus sequence viewed using the CLUSTAL 2.0.10 multiple sequence alignment is shown below.

As demonstrated in Example 1, several peptides were identified and could be used to construct a consensus sequence for an endothelial basement membrane targeting peptide. Useful peptides are those binding to the same epitopes and having at least 70, 80, 90, 95, 98, or 99 percent sequence identity to KIWKLPQ (SEQ ID NO:1), KVWSLPQ (SEQ ID NO:2), KLWVLPK (SEQ ID NO:3), or KIFVWPY (SEQ ID NO:4).

| | | |
|---|---|---|
| C-10 | KIWKLPQ | (SEQ ID NO: 1) |
| A-8 | KVWSLPQ | (SEQ ID NO: 2) |
| C-11 | KLWVLPK | (SEQ ID NO: 3) |
| A-9 | KIFVWPY | (SEQ ID NO: 4) |
| | *:: * | |
| Consensus | KIWVLPQ | (SEQ ID NO: 5) |

As described in more detail below, a fully representative combinatorial library of random heptamers fused to a minor coat protein (pill) of M13 filamentous phage was subjected to five rounds of biopanning against human collagen IV to discover a functional vascular targeting peptide. Fifteen clones per round were randomly sequenced from Round 3 to 5. 100% of the clones in R5 were found to be C-8, HWGSLRA (SEQ ID NO:24). To find similarities to resident basement membrane structures, the pBLAST algorithm (Altschul, et al. (1997) Nucleic Acids Res 25, 3389-3402; Schaffer, et al. (2001) Nucleic Acids Res 29, 2994-3005) was used to search the non-redundant version of the current National Center for Biotechnology Information (NCBI) homo sapiens sequence database against peptides from the screen. Sequences were classified into three groups. The first group consists of peptides with homology to resident basement membrane proteins such as nidogen, serum amyloid P component, gelsolin and laminin (Kalluri, R. (2003) Nat Rev Cancer 3, 422-433) (group A). The second group of peptides was enriched in proline residues, such as Pro-Pro-Ser (PPS) and Pro-Pro-Pro (PPP) runs, which resemble the Gly-Pro-Pro (GPP) motif in the collagen triple helix (Hudson, et al. (1993) J Biol Chem 268, 26033-26036) (group B). The third group consisted of unique peptides with no identifiable relationship with the basement membrane (group C).

23 clones were incubated in triplicate against Matrigel and bovine serum albumin (BSA). No reactivity was observed against BSA compared with the random library (R0). Despite the similarity of the GPP and PP motifs with collagen IV, the peptides in group 2 had less binding affinity compared to Groups A and C, and did not show any detectable binding affinity above the library. Clones A-8, A-9, C-10 and C-11 were the best candidates. The four clones were aligned pairwise using the CLUSTAL 2.0.10 multiple sequence alignment and gave a consensus sequence KIWVLPQ (SEQ ID NO:5), or more stringently, KZWXLPX (SEQ ID NO:6), where Z is a hydrophobic amino acid and X is any amino acid.

The peptides have a variety of uses. These can be fused to other proteins to target the proteins, they can be bound to substrates such as medical devices, nano or microparticles or conjugated to therapeutic, prophylactic or diagnostic agents to target the particles or conjugate to endothelium that has been disrupted or injured, or they can be bound to a material to facilitate adhesion of the material to the disrupted or injured endothelium.

B. Substrates for the Ligands

The endothelial basement membrane targeting peptide ligands can be bound to any substrate, including substrates formed of polymer, metal, ceramic, or combinations thereof, using conventional methods. The ligands can be used to target and/or adhere the materials to disrupted or injured endothelium.

1. Forms of Devices or Substrates

A “microparticle” is a particle having an average diameter on the order of micrometers (i.e., between about 1 micrometer and about 1 mm), while a “nanoparticle” is a particle having an average diameter on the order of nanometers (i.e., between about 1 nm and about 1 micrometer. The particles may be spherical or non-spherical, in some cases. Nanoparticles and microparticles are jointly referred to herein as microparticles or particles unless otherwise specified.

Other device substrates include polymeric or metallic materials used to form catheters or stents. These can also be in the form of films, gels, sponges, or foams, that are applied at the time of surgery, by catheter.

The ligands can also be applied to or bound to microwell plates, slides, tubes, columns, gels, or other means for diagnostic reaction or detection of molecules in samples of tissue or cells, or materials in solution such as a biological sample or library which bind to the ligand.

2. Materials Used to Form Device or Substrate

The particles or substrate may be formed of any suitable material, depending on the application. For example, the particles or substrate may comprise a metal, glass, lipid and/or a polymer. In the preferred embodiment, the particles are formed from biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these. The particles may comprise a hydrogel, such as agarose, collagen, or fibrin.

Non-biodegradable or biodegradable polymers may be used to form the microparticles or substrates. In the preferred embodiment, the microparticles are formed of a biodegradable polymer. In general, synthetic polymers are preferred, although natural polymers may be used and have equivalent or even better properties, especially some of the natural biopolymers which degrade by hydrolysis, such as some of the polyhydroxyalkanoates. Representative synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate. The in vivo stability of the microparticles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

Examples of preferred non-biodegradable polymers include ethylene vinyl acetate, poly(meth)acrylic acid, polyamides, copolymers and mixtures thereof.

In the most preferred embodiment, PLGA is used as the biodegradable polymer. PLGA microparticles are designed to release molecules to be encapsulated or attached over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity and that in turn affects the degradation rate. For example, the hydrophobic poly (lactic acid) (PLA), more hydrophilic poly (glycolic acid) PGA and their copolymers, poly (lactide-co-glycolide) (PLGA) have various release rates. The degradation rate of these polymers, and often the corresponding drug release rate, can vary from days (PGA) to months (PLA) and is easily manipulated by varying the ratio of PLA to PGA.

Liposomes are lipid vesicles composed of concentric phospholipid bilayers which enclose an aqueous interior (Gregoriadis, et al., hit J Pharm 300, 125-30 2005; Gregoriadis and Ryman, Biochem J 124, 58P (1971)). The lipid vesicles comprise either one or several aqueous compartments delineated by either one (unilamellar) or several (multilamellar) phospholipid bilayers (Sapra, et al., Curr Drug Deliv 2, 369-81 (2005)). Liposomes have the ability to form a molecular film on cell and tissue surfaces and are currently being tested as possible therapeutic agents to promote wound healing and healing dry eye as a tear substitute. Clinical studies have proven the efficacy of liposomes as a topical healing agent (Dausch, et al., Klin Monatsbl Augenheilkd 223, 974-83 (2006); Lee, et al., Klin Monatsbl Augenheilkd 221, 825-36 (2004)). More than ten liposomal and lipid-based formulations have been approved by regulatory authorities and many liposomal drugs are in preclinical development or in clinical trials (Barnes, Expert Opin. Pharmacother. (2006) 7, 607-615; Minko, et al. Anticancer Agents Med. Chem (2006) 6, 537-552.

Suitable metallic materials include, but are not limited to, metals and alloys based on titanium (such as nitinol, nickel titanium alloys, thermo-memory alloy materials), stainless steel, tantalum, palladium, zirconium, niobium, molybdenum, nickel-chrome, or certain cobalt alloys including cobalt-chromium and cobalt-chromium-nickel alloys such as Elgiloy® and Phynox®. The particles may include a magnetically susceptible material in some cases, e.g., a material displaying paramagnetism or ferromagnetism. The particles may include iron, iron oxide, magnetite, hematite, or some other compound containing iron. The particles can include a conductive material (e.g., a metal such as titanium, copper, platinum, silver, gold, tantalum, palladium, rhodium, etc.), a semiconductive material (e.g., silicon, germanium, CdSe, CdS, etc.) or a radioopaque material. Other particles include ZnS, ZnO, TiO₂, AgI, AgBr, HgI₂, PbS, PbSe, ZnTe, CdTe, In₂S₃, In₂Se₃, Cd₃P₂, Cd₃As₂, InAs, or GaAs.

In some cases, the particles may comprise a ceramic such as tricalcium phosphate, hydroxyapatite, fluorapatite, aluminum oxide, or zirconium oxide. Suitable ceramic materials include, but are not limited to, oxides, carbides, or nitrides of the transition elements such as titanium oxides, hafnium oxides, iridium oxides, chromium oxides, aluminum oxides, and zirconium oxides. Silicon based materials, such as silica, may also be used.

C. Molecules to be Conjugated to the Ligands or Attached to Particles Targeted by the Ligands

There are two principle groups of molecules to be attached to the ligand or targeted particle, either directly or via a coupling molecule: targeting or attachment molecules and therapeutic, nutritional, diagnostic, prophylactic or barrier agents (as noted above, these are jointly referred to as pharmaceutical agents). These can be coupled, conjugated or encapsulated using standard techniques. The targeting molecule or therapeutic molecule to be delivered can be coupled directly to the polymer or to a material which is incorporated into the polymer, as discussed below. Proteins, peptides, carbohydrates, polysaccharides, nucleic acid molecules, and organic molecules, as well as diagnostic agents, can be delivered.

The materials described above can be used to deliver or adhere at a site where the endothelium has been disrupted or injured, any therapeutic, prophylactic or diagnostic agent. A therapeutic agent may be a pharmacologically active agent or it may be a molecule that forms a barrier to prevent or suppress leakage through the permeabilized endothelium. Representative barrier agents include nano or microparticles, polymers such as alginates, hyaluronates, collagens, glycoproteins, PEG-PLGA polymers (FOCALSEAL®), PEO-PPG block copolymers (PLURONICS®), self-assembling peptides such as those described in US patent application Nos. 20080091233 and 20090111734. Representative pharmacological agents include anti-angiogenic agents and agents which cause vascular regrowth. The preferred materials to be incorporated are drugs such as anti-cancer (referred to herein as “chemotherapeutics”, including cytotoxic drugs such as doxorubicin, cyclosporine, mitomycin C, cisplatin and carboplatin, BCNU, 5FU, methotrexate, adriamycin, camptothecin, and taxol), antibodies and bioactive fragments thereof (including humanized, single chain, and chimeric antibodies), peptide drugs, anti-inflammatories, and oligonucleotide drugs (including DNA, RNAs, antisense, aptamers, ribozymes, external guide sequences for ribonuclease P, and triplex forming agents).

Particularly preferred drugs to be delivered include anti-angiogenic agents, antiproliferative and chemotherapeutic agents such as taxol and rampamycin. Incorporated into microparticles, these agents may be used to treat cancer or eye diseases, or prevent restenosis following administration into the blood vessels. Exemplary diagnostic materials include paramagnetic molecules, fluorescent compounds, magnetic molecules, and radionuclides.

The microparticles may be further modified by attachment of one or more different molecules, such as additional targeting and/or attachment molecules, and/or therapeutic, nutritional, diagnostic or prophylactic agents. A targeting molecule is a substance which will direct the microparticle to a receptor site on a selected cell or tissue type, can serve as an attachment molecule, or serve to couple or attach another molecule.

Targeting molecules can be proteins, peptides, nucleic acid molecules, saccharides or polysaccharides that bind to a receptor or other molecule on the surface of a targeted cell. These may be in addition to the peptide ligands which target the particles to endothelial basement membrane. The degree of specificity can be modulated through the selection of the targeting molecule. For example, antibodies are very specific. These can be polyclonal, monoclonal, fragments, recombinant, or single chain, many of which are commercially available or readily obtained using standard techniques.

II. Methods of Manufacture of Particles and Substrates having Peptide Bound Thereto

A. Methods for Making Nano and Microparticles

The agents may be incorporated into, onto, or coupled to nano or microparticles, preferably formed of biocompatible, biodegradable polymers. As used herein, nanoparticles have a diameter of less than one micron; microparticles have a diameter of greater than one micron, typically less than 500 microns, most preferably for injection in the range of one to 10 microns. Unless specified, “particles” refers to both nano and microparticles.

Two parameters of drug loading and release are important for drug efficacy. Increased drug loading into the particle core tends to reduce overall stability, giving an undesired burst release effect and reduced efficacy. Larger particles have slower release profiles, but when systemically administered are more readily detected and cleared from circulation, resulting in a lack of efficacy. For vascular targeting, since small particles show improved vessel adhesion and retention, incorporating slow-eluting conjugates into the nanoparticle design allows for: (i) improved drug encapsulation; (ii) sub-100 nm NPs for vascular targeting; and (iii) sustained drug release over two weeks.

Nanoparticles can be prepared using many known methods. In the preferred embodiment, the nanoparticles are prepared as described by Zhang, et al., ACS Nano. (2008) 2(8):1696-702. This method prepares a lipid-polymer hybrid nanoparticle with high drug encapsulation yield, tunable and sustained drug release profile, excellent serum stability, and potential for differential targeting of cells or tissues. The nanoparticles include three distinct functional components: (i) a hydrophobic polymeric core where poorly water-soluble drugs can be encapsulated; (ii) a hydrophilic polymeric shell with antibiofouling properties to enhance nanoparticle stability and systemic circulation half-life; and (iii) a lipid monolayer at the interface of the core and the shell that acts as a molecular fence to promote drug retention inside the polymeric core, thereby enhancing drug encapsulation efficiency, increasing drug loading yield, and controlling drug release. The NP is prepared by self-assembly through a single-step nanoprecipitation method in a reproducible and predictable manner.

In the preferred embodiment, a hybrid NP system was engineered to have a hydrophobic drug-eluting core, a hydrophilic polymeric shell, and a lipid monolayer, as described by Chan, et al. (2009) Biomaterials 30, 1627-1634. Poly(ethylene glycol) (PEG) covalently conjugated to 1,2-distearoyl-sn-glycero-3-phosphoethanolamine (DSPE) was used to form the hydrophilic polymeric shell. To complete the lipid monlayer, soybean lecithin, which is considered Generally Regarded as Safe (GRAS), was used to form the core-shell interface.

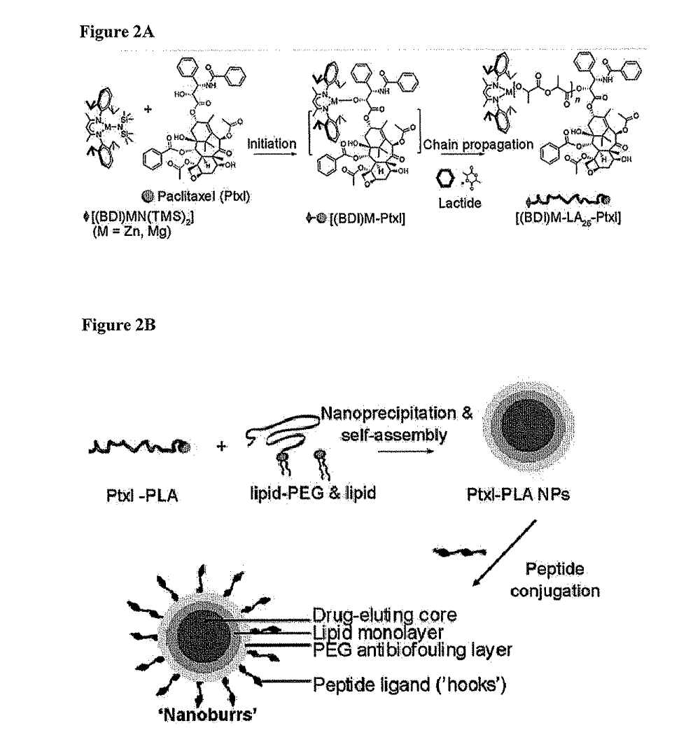

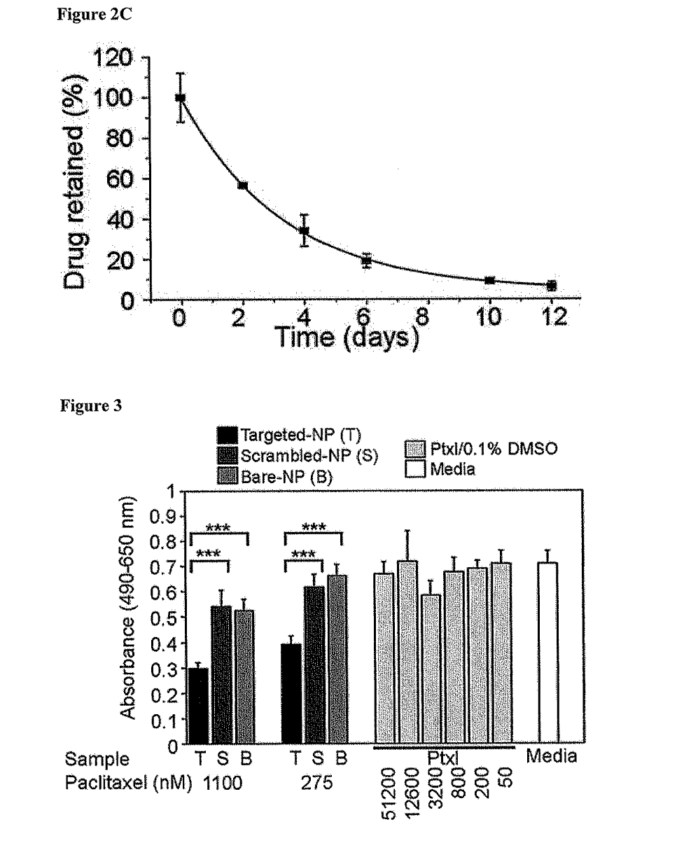

As demonstrated by the examples, the nanoparticulate (NP) system designed with an approximately 60 nm core-shell hybrid NP system consisting of a polymeric core, a lipid interface and a PEG corona formed of poly(lactic acid) (PLA) conjugates of paclitaxel made by a modified drug-alkoxide ring-opening strategy (Chamberlain, et al. (2001) J Am Chem Soc 123, 3229-3238; Dechy-Cabaret, et al. (2004) Chem Rev 104, 6147-6176), allowed for controlled drug release by gradual ester hydrolysis despite the large surface area and short diffusion distances in sub-100 nm particles. For the hydrophobic drug-eluting core, drug-polylactide conjugates were synthesized by a drug/alkoxide-initiated ring-opening polymerization strategy. In FIG. 2B, nanoparticle synthesis is illustrated in which the core (Ptxl-PLA conjugate) and shell (lipid and lipid-PEG) were integrated via nanoprecipitation and self-assembly. The NPs were functionalized with ligands (Peer, et al. (2007) Nat Nanotechnol 2, 751-760; Langer, R. (1998) Nature 392, 5-10) to increase targeting specificity across a range of diseases in a consistent and reproducible manner. The KLWVLPK peptide was conjugated via a C-terminal GGGC (SEQ ID NO:28) linker to DSPE-PEG-maleimide using maleimide-thiol conjugation chemistry. Drug elution rates can be further controlled by varying lactide/drug ratios during ring-opening polymerization, resulting in different PLA chain lengths attached to the drug.

In addition to the preferred method described in the examples for making a high density microparticle, there may be applications where microparticles can be fabricated from different polymers using different methods.

Solvent Evaporation. In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid microparticles. The resulting microparticles are washed with water and dried overnight in a lyophilizer. Microparticles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

Hot Melt Microencapsulation. In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microparticles are washed by decantation with petroleum ether to give a free-flowing powder. Microparticles with sizes between 0.5 to 1000 microns are obtained with this method. The external surfaces of spheres prepared with this technique are usually smooth and dense. This procedure is used to prepare microparticles made of polyesters and polyanhydrides. However, this method is limited to polymers with molecular weights between 1,000-50,000.

Solvent Removal. This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make microparticles from polymers with high melting points and different molecular weights. Microparticles that range between 1-300 microns can be obtained by this procedure. The external morphology of spheres produced with this technique is highly dependent on the type of polymer used.

Spray-Drying. In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm. Microparticles ranging between 1-10 microns are obtained with a morphology which depends on the type of polymer used.

Hydrogel Microparticles. Microparticles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath for twenty to thirty minutes in order to allow sufficient time for gelation to occur. Microparticle particle size is controlled by using various size extruders or varying either the nitrogen gas or polymer solution flow rates. Chitosan microparticles can be prepared by dissolving the polymer in acidic solution and crosslinking it with tripolyphosphate. Carboxymethyl cellulose (CMC) microparticles can be prepared by dissolving the polymer in acid solution and precipitating the microparticle with lead ions. In the case of negatively charged polymers (e.g., alginate, CMC), positively charged ligands (e.g., polylysine, polyethyleneimine) of different molecular weights can be ionically attached.

B. Methods for Coupling Peptides to Surfaces

Methods for coupling peptides to metals, ceramics and polymers are well known. In a preferred embodiment, peptides are coupled to nanoparticles as described by Gu, et al., in Methods Mol. Biol. (2009) 544:589-5999, which describes the preparation of drug-encapsulated nanoparticles formulated with biocompatible and biodegradable poly(D:,L:-lactic-co-glycolic acid)-block-poly(ethylene glycol) (PLGA-b-PEG) copolymer and surface functionalized with the A10 2-fluoropyrimidine ribonucleic acid aptamers.

Other methods are well known. Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways. The first is during the preparation of the microparticles, for example during the emulsion preparation of microparticles by incorporation of stablizers with functional chemical groups. A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after prepartion. This second class also includes a process whereby amphiphilic molecules such as fatty acids, lipids or functional stabilizers may be passively adsorbed and adhered to the particle surface, thereby introducing functional end groups for tethering to ligands. For example, approaches to introduce functionality into PLGA surfaces include synthesis of PLGA copolymers with amine (Lavik et al J Biomed Mater Res 2001; 58(3):291-4; Caponetti et al. J Pharm Sci 1999; 88(1):136-41) or acid (Caponetti et al J Pharm Sci 1999; 88(1):136-41) end groups followed by fabrication into particles. Another approach involves the blending or adsorption of functional polymers such as polylysine (Faraasen et al. Pharm Res 2003; 20(2):237-46; Zheng et al. Biotechnology Progress 1999; 15(4):763-767) or poly(ethylene-alt-maleic acid) (PEMA) (Keegan et al. Macromolecules 2004) or PEG (Muller J Biomed Mater Res 2003; 66A(1):55-61) into PLGA and forming particles and matrices from these blends (Zheng, et al. 1999; Keegan, 2004; Park et al. J Biomater Sci Polym Ed 1998; 9(2):89-110; Croll Biomacromolecules 2004; 5(2):463-73; Cao et al. Methods Mol Biol 2004; 238:87-112). Plasma treatment of the PLGA matrix has also been proposed for the purpose of modifying its surface properties and introducing hydrophilic functional groups into the polymer (Yang et al. J Biomed Mater Res 2003; 67A(4):1139-47; Wan et al., Biomaterials 2004; 25(19):4777-83). The most widely used coupling group is poly(ethylene glycol) (PEG), because this group creates a hydrophilic surface that facilitates long circulation of the nanoparticles.

Incorporating ligands in liposomes is easily achieved by conjugation to the phospholipid head group, in most cases phosphotidylethanolamine (PE), and the strategy relies either on a preinsertion of the functionalized lipid or post insertion into a formed liposome. Functionality can also be introduced by incorporating PEG with functional endgroups for coupling to target ligands.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

A useful coupling procedure for attaching ligands with free thiol groups to polymers involves the use of polymers with maleimide end-groups. This method is useful for attaching peptides, nucleic acids and antibodies which are modified to contain cysteines (thiol groups) for conjugation to maleimide. Briefly, the activation involves reduction of disulfide bonds formed between cysteine thiol groups of ligands by a reducing agent, TCEP ((tris(2-carboxyethyl)phosphine)) in a oxygen-free environment, then adding the polymer (with maleimide end-group) to the reduced ligand. Activation and coupling take place at 1-10 mM EDTA at pH 6.5-7.5. The linkage is a covalent and stable linkage in the pH range of 1-8 once conjugation has taken place.

Any suitable coupling method known to those skilled in the art for the coupling of ligands and polymers with double bonds, including the use of UV crosslinking, may be used for attachment of molecules to the polymer. Coupling is preferably by covalent binding but it may also be indirect, for example, through a linker bound to the polymer or through an interaction between two molecules such as strepavidin and biotin. It may also be by electrostatic attraction by dip-coating.

The molecules to be delivered can also be encapsulated into the polymer using double emulsion solvent evaporation techniques, such as that described by Luo et al., Controlled DNA delivery system, Phar. Res., 16: 1300-1308 (1999).

### III. Methods of Administration and Treatment

The vascular system has the critical function of supplying tissues with nutrients and clearing waste products. To accomplish these goals, the vasculature must be sufficiently permeable to allow the free, bidirectional passage of small molecules and gases and, to a lesser extent, of plasma proteins. Permeability is an extremely complicated process that, however defined, is affected by many different variables. These include the intrinsic properties of the different types of microvessels involved (capillaries, venules, mother vessels (MV)); the size, shape, and charge of extravasating molecules; the anatomic pathways molecules take in crossing the endothelial cell barrier; the time course over which permeability is measured; and the animals and vascular beds that are being investigated. Vascular permeability is dramatically increased in acute and chronic inflammation, cancer, and wound healing. This hyperpermeability is mediated by acute or chronic exposure to vascular permeabilizing agents, particularly vascular permeability factor/vascular endothelial growth factor (VPF/VEGF, VEGF-A). Three distinctly different types of vascular permeability can be distinguished, based on the different types of microvessels involved, the composition of the extravasate, and the anatomic pathways by which molecules of different size cross-vascular endothelium. These are the basal vascular permeability (BVP) of normal tissues, the acute vascular hyperpermeability (AVH) that occurs in response to a single, brief exposure to VEGF-A or other vascular permeabilizing agents, and the chronic vascular hyperpermeability (CVH) that characterizes pathological angiogenesis. Nagy, et al., Angiogenesis 11(2):109-119 (June 2008).

Alternatively permeability can be measured as net amount of a solute, typically a macromolecule such as plasma albumin, that has crossed a vascular bed and accumulated in the interstitium in response to a vascular permeabilizing agent or at a site of pathological angiogenesis. Generally speaking, the vessels involved are not of a single type, and the measurements made combine together all of the factors, both intrinsic properties of the blood vessels as well as extrinsic properties such as blood flow, that regulate extravasation. This can be determined using the Miles assay or one of its variants. Typically, a dye such as Evan's blue that binds noncovalently to albumin is injected intravenously and its accumulation is measured at some later time at a skin test site, in a tumor, or in other tissues of interest. Permeability is defined as the amount of albumin-dye complex that is present at some time (often 30 min) after Evan's blue injection. The intensity of local bluing observed visually provides sufficient information for some purposes. Quantitative measurements also can be made by extracting the dye from tissues and measuring it spectrophotometrically.

The materials described herein may be administered systemically for any disorder or diseases where the endothelial lining is compromised, for example, oncologic diseases, cardiovascular inflammatory disease, ophthalmic diseases, the gastrointestinal and pulmonary tracts of premature babies, sepsis, and transplantation. A preferred application is in the delivery of anti-proliferative agents to the lining of blood vessels following angioplasty, transplantation or bypass surgery to prevent or decrease restenosis, and in cancer therapy. In still another application, the materials are administered to the eye, to treat ophthalmic disorders such as macular degeneration. In another application, barrier materials are administered to the gastrointestinal or pulmonary tracts of premature babies or patients with sepsis.

A. Restenosis and Transplantation

Percutaneous transluminal coronary angioplasty (PTCA) is a procedure in which a small balloon-tipped catheter is passed down a narrowed coronary artery and then expanded to re-open the artery. It is currently performed in approximately 250,000-300,000 patients each year. The major advantage of this therapy is that patients in which the procedure is successful need not undergo the more invasive surgical procedure of coronary artery bypass graft. A major difficulty with PTCA is the problem of post-angioplasty closure of the vessel, both immediately after PTCA (acute reocclusion) and in the long term (restenosis).

The mechanism of acute reocclusion appears to involve several factors and may result from vascular recoil with resultant closure of the artery and/or deposition of blood platelets along the damaged length of the newly opened blood vessel followed by formation of a fibrin/red blood cell thrombus. Restenosis (chronic reclosure) after angioplasty is a more gradual process than acute reocclusion: 30% of patients with subtotal lesions and 50% of patients with chronic total lesions will go on to restenosis after angioplasty. Although the exact hormonal and cellular processes promoting restenosis are still being determined, it is currently understood that the process of PTCA, besides opening the artherosclerotically obstructed artery, also injures resident coronary arterial smooth muscle cells (SMC). In response to this injury, adhering platelets, infiltrating macrophages, leukocytes, or the smooth muscle cells (SMC) themselves release cell derived growth factors with subsequent proliferation and migration of medial SMC through the internal elastic lamina to the area of the vessel intima. Further proliferation and hyperplasia of intimal SMC and, most significantly, production of large amounts of extracellular matrix over a period of 3-6 months, results in the filling in and narrowing of the vascular space sufficient to significantly obstruct coronary blood flow.

The treatment of restenosis requires additional, generally more invasive, procedures, including coronary artery bypass graft (CABG) in severe cases. Consequently, methods for preventing restenosis, or treating incipient forms, are being aggressively pursued. One possible method for preventing restenosis is the administration of anti-inflammatory compounds that block local invasion/activation of monocytes thus preventing the secretion of growth factors that may trigger SMC proliferation and migration. Other potentially anti-restenotic compounds include antiproliferative agents that can inhibit SMC proliferation, such as rapamycin and paclitaxel. Rapamycin is generally considered an immunosuppressant best known as an organ transplant rejection inhibitor. However, rapamycin is also used to treat severe yeast infections and certain forms of cancer. Paclitaxel, known by its trade name Taxol®, is used to treat a variety of cancers, most notably breast cancer.

However, anti-inflammatory and antiproliferative compounds can be toxic when administered systemically in anti-restenotic-effective amounts. Furthermore, the exact cellular functions that must be inhibited and the duration of inhibition needed to achieve prolonged vascular patency (greater than six months) are not presently known. Moreover, it is believed that each drug may require its own treatment duration and delivery rate. Therefore, in situ, or site-specific drug delivery using anti-restenotic coated stents has become the focus of intense clinical investigation. Recent human clinical studies on stent-based delivery of rapamycin and paclitaxel have demonstrated excellent short-term anti-restenotic effectiveness. Stents, however, have drawbacks due to the very high mechanical stresses, the need for an elaborate procedure for stent placement, and manufacturing concerns associated with expansion and contraction.

One of the most promising applications for targeted drug delivery using nanoparticles is in local application using interventional procedures such as catheters. Potential applications have focused on intra-arterial drug delivery to localize therapeutic agents in the arterial wall to inhibit restenosis (Labhasetwar, et al. J Pharm Sci 87, 1229-1234 (1998); Song, et al. J Control Release 54, 201-211 (1998)). Drug loaded nanoparticles are delivered to the arterial lumen via catheters and retained by virtue of their size, or they may be actively targeted to the arterial wall by non-specific interactions such as charged particles or particles that target the extracellular matrix. Surface-modified nanoparticles, engineered to display an overall positive charge facilitated adhesion to the negatively charged arterial wall and showed a 7 to 10-fold greater arterial localized drug levels compared to the unmodified nano-particles in different models. This was demonstrated to have efficacy in preventing coronary artery restenosis in dogs and pigs (Labhasetwar, et al. J Pharm Sci 87, 1229-1234 (1998)). Nanoparticles loaded with dexamethasone and passively retained in arteries showed reduction in neointimal formation after vascular injury (Guzman, et al. Circulation 94, 1441-1448 (1996)).

The microparticles (and/or nanoparticles) can be used in these procedures to prevent or reduce restenosis. Microparticles can be delivered at the time of bypass surgery, transplant surgery or angioplasty to prevent or minimize restenosis. The microparticles can be administered directly to the endothelial surface as a powder or suspension, during or after the angioplasty, or coated onto or as a component of a stent which is applied at the time of treatment. The microparticles can also be administered in conjunction with coronary artery bypass surgery. In this application, particles are prepared with appropriate agents such as anti-inflammatories or anti-proliferatives. These particles are made to adhere to the outside of the vessel graft by addition of adhesive ligands as described above. A similar approach can be used to add anti-inflammatory or immunosuppressant loaded particles to any transplanted organs or tissues.

In this embodiment, the drug to be delivered is preferably an anti-proliferative such as taxol, rapamycin, sirulimus, or other antibiotic inhibiting proliferation of smooth muscle cells, alone or in combination with an anti-inflammatory, such as the steroidal anti-inflammatory dexamethasone. The drug is encapsulated within and optionally also bound to the microparticles.

The targeted drugs can be delivered at the time of angioplasty: locally by intraarterial delivery, or systemically by intravenous delivery. The targeted drugs can also be delivered as second or third doses (and more) by intravenous delivery at later time points (days or weeks or months or even years). The drug dose depends on the anti-proliferative drug used, which will be readily determined by those skilled in the art based on known effective dosages.

B. Treatment of Tumors

Passive delivery may also be targeted to tumors. Aggressive tumors inherently develop leaky vasculature with 100 to 800 nm pores due to rapid formation of vessels that must serve the fast-growing tumor. This defect in vasculature coupled with poor lymphatic drainage serves to enhance the permeation and retention of nanoparticles within the tumor region. During tumor development, the neovasculature formed as a result of increased demand for oxygen and nutrients have been extensively characterized to be leaky and dysfunctional, resulting in many regions with exposed basement membrane.

Passive targeting will probably dominate in the NP size range of 100-500 nm, but ultimately, nanoparticle retention after 24-72 h due to targeting would probably be greater than non-targeted NP groups. The targeted drug may be given as a course of chemotherapy every two days over two weeks (in animal xenograft studies) by systemic intravenous delivery. Preferred drugs include anti-proliferative drugs such as those used for restenosis, for example, taxanes (docetaxel and paclitaxel) and rapamycin, and also others such as cisplatin.

The particles described herein should be efficacious in the treatment of tumors, especially those where targeting is beneficial and delivery of high doses of chemotherapeutic desirable. An important feature of targeted particle delivery is the ability to simultaneously carry a high density of drug while displaying ligands on the surface of the particle.

C. Ophthalmic Disorders

Macular degeneration (MD) is a chronic eye disease that occurs when tissue in the macula, the part of the retina that is responsible for central vision, deteriorates. Degeneration of the macula causes blurred central vision or a blind spot in the center of your visual field. Macular degeneration occurs most often in people over 60 years old, in which case it is called Age-Related Macular Degeneration (ARMD) or (AMD). AMD is the leading cause of blindness in the United States and many European countries. About 85-90% of AMD cases are the dry, atrophic, or nonexudative form, in which yellowish spots of fatty deposits called drusen appear on the macula. The remaining AMD cases are the wet form, so called because of leakage into the retina from newly forming blood vessels in the choroid, a part of the eye behind the retina. Normally, blood vessels in the choroid bring nutrients to and carry waste products away from the retina. Sometimes the fine blood vessels in the choroid underlying the macula begin to proliferate, a process called choroidal neovascularization (CNV). When those blood vessels proliferate, they leak, causing damage to cells in the macula often leading to the death of such cells. The neovascular “wet” form of AMD is responsible for most (90%) of the severe loss of vision. There is no cure available for “wet” or “dry” AMD.

Treatments for wet AMD include photocoagulation therapy, photodynamic therapy, and transpupillary thermotherapy. Other potential treatments for “wet” AMD that are under investigation include angiogenesis inhibitors, such as anti-VEGF antibody, and anti-VEGF aptamer (NX-1838), integrin antagonists to inhibit angiogenesis has also been proposed, and PKC412, an inhibitor of protein kinase C. Cytochalasin E (Cyto E), a natural product of a fungal species that inhibits the growth of new blood vessels is also being investigated to determine if it will block growth of abnormal blood vessels in humans. The role of hormone replacement therapy is being investigated for treatment of AMD in women.

D. Endothelium Dysfunction in Sepsis

The vascular endothelium regulates blood vessel tone, vascular permeability, coagulation, angiogenesis, white blood cell and platelet activity, and phagocytosis of bacteria. The endothelium produces a number of vasoactive substances including Nitric Oxide, Endothelium-derived relaxing factor (EDRF), Prostacyclin, and Endothelin-1. Nitric Oxide (NO) is produced from L-arginine by nitric oxide synthetase (NOS).

Activity is via cGMP and it effects vasodilatation and inhibits platelet aggregation. Endothelin-1 is a potent vasoconstrictor, increasing circulating levels in cardiogenic shock and following severe trauma.

The dominant haemodynamic feature in septic shock is peripheral vascular failure, leading to persistent hypotension resistant to vasoconstrictors. This is due to myocardial edema and microcirculatory changes leading to capillary leak syndrome. Vasodilation causes maldistribution of flow, A-V shunting, increased capillary permeability and interstitial edema, and decreased oxygen extraction.

The process of microcirculatory failure in shock includes the following steps:

### Stage 1: Compensation

The pre capillary arterioles and post capillary venules vasoconstrict: this helps maintain systemic blood pressure. There is increased hydrostatic pressure in the capillaries, consequently fluid is “sucked”/sequestered from the interstitium. This is known as “transcapillary refill”. This leads to restoration of circulating volume, along with the renin-angiotensin-aldosterone axis.

### Stage 2: Decompensation

The accumulation of hydrogen ions, lactic acid, increased PaCO2 & vasoactive substances, occurs. Precapillary sphincters relax but, the post capillary venules become unresponsive and vasoconstrict. Blood “sieves” out of the capillary bed, resulting in interstitial oedema/haemoconcentration/increased blood viscosity. Intravascular dehydration results, with procoagulant effects: with platelet activation and clot formation in the capillary bed. Antigen-antibody complexes are laid down, endotoxin is released, tissue thromboplastin is released, the intrinsic pathway is activated, resulting in disseminated intravascular coagulation (“DIC”), with cell damage due to thrombosis and ischaemia and cell compression by interstitial edema. Ultimately, there is consumption of clotting factors and abnormal bleeding. Capillary endothelial injury follows, with microemboli, release of vasoactive components, complement activation, and extravascular migration of leucocytes. Capillary permeability is increased so that fluid is lost into the interstitial space, leading to hypovolaemia/interstitial oedema/organ dysfunction. Reperfusion of the microcirculation leads to the generation of large quantities of oxygen free radicals leading to tissue damage, particularly to the gut mucosa.

The conjugates described herein may be systemically applied at any point in sepsis in an effort to decrease endothelial permeability or organ failure, either by selective targeted delivery of barrier conjugates or delivery of drugs.

E. Premature Infants

Preterm newborns who require mechanical ventilation and supplemental oxygen are at risk for bronchopulmonary dysplasia (BPD), a chronic lung disease of newborn infants associated with significant mortality and morbidity. BPD in the postsurfactant era is seen mainly in very low birthweight infants and affects 30% of infants born between 24 and 28 weeks of gestation, many of whom will require long-term respiratory support. Although the microvasculature in ventilated preterm lungs is quantitatively nearly normal, there are angioarchitectural abnormalities compared with age-matched nonventilated control lungs. Normal human lungs at term (36-40 wk of gestation) display thin alveolar septa with abundant secondary crest formation, characteristic of the early alveolar stage of lung development. Within the complex alveolar septa of normal lungs, the microvasculature forms a delicate network, characterized by extensive capillary sprouting. In contrast, the pulmonary microvasculature of long-term ventilated preterm infants at the same corrected postmenstrual age (36-40 wk) retains the vascular pattern of canalicular/saccular lungs, characterized by a persistent dual capillary pattern and primitive, nonbranching vessels.

Intestinal permeability is higher in immature neonates than in older children and adults. Preterm infants born at less than 33 weeks of gestation have higher serum concentrations of β-lactoglobulin than do term infants given equivalent milk feedings. The permeability of the preterm human intestine to intact carbohydrate markers such as lactulose exhibits a developmental pattern of increased permeability with maturation. Little is currently known about the maturation of tight junction proteins such as occludin and claudins, which constitute the major paracellular barrier of the epithelium.

Similar to sepsis and adult respiratory stress syndrome, necrotizing enterocolitis (“NEC”) involves a final common pathway that includes the endogenous production of inflammatory mediators involved in the development of intestinal injury. Endotoxin lipopolysaccharide, platelet-activating factor (PAF), tumor necrosis factor, and other cytokines together with prostaglandins and leukotrienes and nitric oxide are thought to be involved in the final common pathway of NEC pathogenesis.

F. Diagnostic Applications

The conjugates can be used for diagnostic purposes, to measure permeability, to detect or quantitate vasoactive compounds, and to detect areas of disrupted endothelium. Diagnostic agents include radiolabeled ligands, fluorescent ligands, and radioopaque ligands.

### EXAMPLES

The present invention will be further understood by reference to the following non-limiting examples.

### Example 1

### Identification of Peptides Specifically Binding to Human Collagen IV

Materials and Methods

Human collagen IV, human collagen I and Matrigel™ growth factor reduced LDEV free were purchased from BD Biosciences (San Jose, Calif.). Soybean lecithin was purchased from Alfa Aeser (Ward Hill, Mass.). 1,2-Distearoyl-sn-Glycero-3-Phosphoethanolamine-N-[Maleimide (PolyethyleneGlycol) 2000] (ammonium salt) (DSPE-PEG-maleimide) and 1,2-Distearoyl-sn-Glycero-3-Phosphoethanolamine-N-[Methoxy (PolyethyleneGlycol) 2000] (ammonium salt) (DSPE-PEG) was purchased from Avanti Polar Lipids (Alabaster, Ala.). Poly(_{D,L}-lactic-co-glycolic acid) (PLGA) polymers (inherent viscosity: 0.19 dl/g) were purchased from Durect Corporation (Cupertino, Calif.). All peptides were custom synthesized by GenScript (Piscataway, N.J.) and purified by reverse-phase high-performance liquid chromatography and mass spectral analysis with >95% purity. Peptides were synthesized with a linker sequence (GGGC, SEQ ID NO:28) at the carboxyl terminus for maleimide-thiol coupling. Alexa Fluor 647 hydrazide tris(triethylammonium) salt was purchased from Invitrogen (Carlsbad, Calif.). Peptides were also amidated at the C-terminus for biological function as they are N-terminally displayed peptides with C-termini linked to the rest of the phage.

Screening of phage display peptide library. The Ph.D.-7 phage library was obtained from New England Biolabs (Beverly, Mass.). Approximately 10 μg/mL human collagen IV in 0.1 M NaHCO₃, pH 8.6 was coated onto a 96-well EIA/RIA high binding plate (Corning Life Sciences, Lowell, Mass.) overnight at 4° C. for biopanning according to the manufacturer's instructions. In R2 to R5, the Tween®-20 concentration was raised to 0.5%, and the collagen IV enriched phage pool from R1 was subtractively panned against human collagen I for 1 h at RT to reduce collagen 1 binding interference prior to biopanning against collagen IV. In R5, 1 μg/mL collagen IV coated plates were used for increased stringency. 15 clones per round were randomly picked from R3 to R5 for DNA sequencing and further analysis.

Results

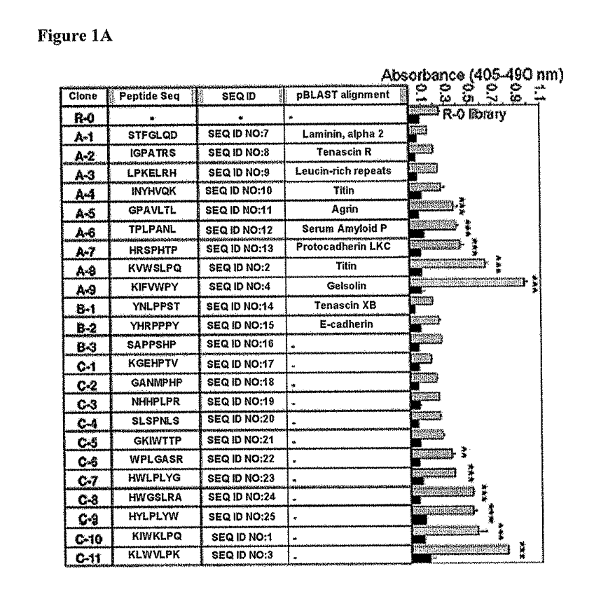

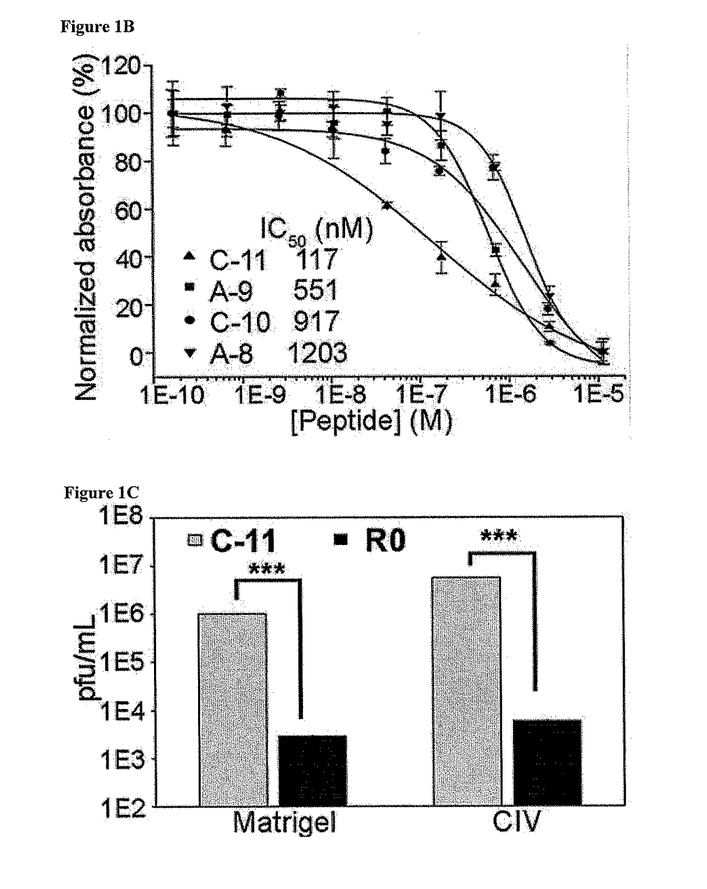

FIG. 1 is a table and graph showing the identification and characterization of peptides for targeting to injured vasculature. 23 phage clones from Rounds 3-5 of the phage display screen were divided into three groups: Group A: Peptide sequences which show homology to resident basement membrane proteins or contain collagen binding-motifs analyzed by pBLAST against the NCBI homo sapiens non-redundant protein sequence database. Group B: Sequences resembling the collagen IV Gly-Pro-Pro (GPP) triple helix. Group C: Sequences with no identifiable relationship to resident basement membrane structures. The clones were tested against the random library (R0) for binding to Matrigel (lighter shaded bars) or BSA (black bars). Bound phages were labeled with peroxidase-conjugated anti-M13 monoclonal antibodies (mAbs), and ABTS absorbance at 405 nm was read against a reference wavelength of 490 nm (mean±s.d., n=3). (**) P<0.01; (***) P<0.001, all compared with R0 (one-way analysis of variance with Tukey post-hoc test).

The alignment and consensus sequence viewed using the CLUSTAL 2.0.10 multiple sequence alignment is shown below.

| C-10 | KIWKLPQ | (SEQ ID NO: 1) |
|---|---|---|
| A-8 | KVWSLPQ | (SEQ ID NO: 2) |
| C-11 | KLWVLPK | (SEQ ID NO: 3) |
| A-9 | KIFVWPY | (SEQ ID NO: 4) |
| | *:: * | |
| Consensus | KIWVLPQ | (SEQ ID NO: 5) |

FIG. 1B is a graph of the sequence-specific competition binding assays of phage clones A-8, A-9, C-10 and C-11 against synthetic peptide equivalents to Matrigel. IC₅₀ values were determined (and normalized on a percentage scale (mean±s.d., n=3, nM): (▴) C-11, 117; (▪) A-9, 551; () C-10, 917; and (▾) A-8, 1203.

FIG. 1C is a graph of the titer count analyses of C-11 compared to R0 on Matrigel and collagen IV. Titers of eluted phages were averaged to give the p.f.u./mL (mean±s.d., n=3). (***) P<0.001 by a paired two-sample Student's t-test. Values were comparable for collagen IV and Matrigel.

To discover a functional vascular targeting peptide, a fully representative combinatorial library of random heptamers fused to a minor coat protein (pill) of M13 filamentous phage was subjected to five rounds of biopanning against human collagen IV. Fifteen clones per round were randomly sequenced from Round 3 to 5 (R3-R5) (FIG. 1A), and in R5, 100% of the clones were found to be C-8, HWGSLRA. To find similarities to resident basement membrane structures, the pBLAST algorithm (Altschul, et al. (1997) Nucleic Acids Res 25, 3389-3402; Schaffer, et al. (2001) Nucleic Acids Res 29, 2994-3005) was used to search the non-redundant version of the current National Center for Biotechnology Information (NCBI) homo sapiens sequence database against peptides from the screen. Sequences were classified into three groups. The first group consists of peptides with homology to resident basement membrane proteins such as nidogen, serum amyloid P component, gelsolin and laminin (Kalluri, R. (2003) Nat Rev Cancer 3, 422-433). The second group of peptides was enriched in proline residues, such as Pro-Pro-Ser (PPS) and Pro-Pro-Pro (PPP) runs, which resemble the Gly-Pro-Pro (GPP) motif in the collagen triple helix (Hudson, et al. (1993) J Biol Chem 268, 26033-26036). Finally, the third group consists of unique peptides with no identifiable relationship with the basement membrane.

It has been discussed in the literature that penultimate rounds of biopanning may be a rich source of phage binders suspected to be lost due to reduced fitness. Possible reasons include reduced infectivity rates of phages for their Escherichia coli hosts due to low pH elution, disulfide-bond formation between cysteine containing phages resulting in the rarity of cysteine-rich sequences, faster growth rates of certain clones, or simply a founder effect when a fraction of amplified phages are input into the next round of biopanning. In FIG. 1A, a binding experiment was performed to examine the binding capacities of sequenced clones against the resultant C-8 clone from R5. 23 clones were incubated in triplicate against Matrigel and bovine serum albumin (BSA). The phages were ranked according to their absorbance values indicating binding capacity to Matrigel. No reactivity was observed against BSA compared with the random library (R0). Despite the similarity of the GPP and PP motifs with collagen IV, Group B peptides had less binding affinity compared to Group A and C, and did not show any detectable binding affinity above the library. Clones A-8, A-9, C-10 and C-11 were the best candidates, and it was noted that these four clones resembled each other. The four clones were aligned pairwise using the CLUSTAL 2.0.10 multiple sequence alignment and gave a consensus sequence of KIWVLPQ (SEQ ID NO:5), or more stringently, KZWXLPX (SEQ ID NO:6), where Z is a hydrophobic amino acid and X is any amino acid.

In a sequence-specific competition assay, the context-dependence of the phage towards the peptide-collagen IV binding interaction was investigated. Synthetic peptides modeled after phage clones A-8, A-9, C-10 and C-11 competitively inhibited their cognate phage in a dose-dependent manner on Matrigel-coated surfaces (FIG. 1B). Phage C-11 showed the best peptide competition, suggesting that C-11 binding affinity may represent a specific peptide-collagen IV interaction independent of the phage context. The binding of phage C-11 was investigated in three independent titer count analyses. Phage titers of C-11 were compared against the library (R0) for binding to Matrigel and collagen IV with an initial phage input of 10¹²/mL plaque forming units (p.f.u.). C-11 showed approximately 300-fold Matrigel binding and approximately 900-fold collagen IV binding compared to the library (n=3, p<0.001).

### Example 2

### Preparation of Drug-Polymer Conjugates and NPs

Materials and Methods

Paclitaxel-Polylactide Conjugation

[(BDI)ZnN(TMS)₂] (BDI=2-((2,6-diisopropylphenyl)amino)-4-[((2,6-diisopropylphenyl)imino)-2-pentene, TMS=trimethylsilyl] (6.2 mg, 0.01 mmol) and Ptxl (8.5 mg, 0.01 mmol) were mixed in 0.5 mL anhydrous THF. D,L-Lactide (36 mg, 0.25 mmol) in 2 mL anhydrous THF was added dropwise to initiate polymerization. Lactide was completely consumed within hours at RT and monitored by FTIR or ¹H NMR spectroscopy. The polymerization solution was added to ethyl ether (25 mL) to precipitate out the Ptxl-PLA₂₅ conjugate (˜25 dl-lactide monomer units, 19.2 wt % is Ptxl).

FIG. 2A is a schematic of Ptxl-PLA biomaterial synthesis. Ptxl was mixed with equimolar amounts of [(BDI)ZnN(TMS)₂]; the (BDI)Zn-Ptxl complex formed in situ initiated and completed the polymerization of lactide.

Synthesis and Characterization of Nanoparticles

For the nanoparticle core, Ptxl-PLA₂₅ drug conjugates which have approximately 25 dl-lactide monomer units were synthesized. RP-HPLC analysis of Ptxl against Ptxl-PLA₂₅ conjugates confirmed identity. FIG. 2B is a schematic of nanoparticle synthesis by nanoprecipitation and self-assembly. Ptxl-PLA in acetone was added dropwise to a heated lipid solution, vortexed vigorously, allowed to self-assemble for 2 h, followed by ultrafiltration and resuspension in PBS buffer to form nanoparticles (NPs). The nanoparticles were peptide-functionalized using maleimide-thiol chemistry. The nanoparticles have a drug-eluting polymeric core, a lipid monolayer, a PEG antibiofouling layer, and peptide ligands (‘hooks’) to adhere to the exposed basement membrane during vascular injury.

A 3 mL DSPE-PEG/lecithin mixture in 4% ethanol containing 0.170 mg DSPE-PEG-Maleimide/DSPE-PEG (1:4 molar ratio) and 0.080 mg lecithin was heated for 3 mM above the lipid phase transition temperature to 68° C. under gentle stirring. 1 mg of Ptxl-PLA in acetone (1 mg/mL) was added dropwise at 1 mL/min. The solution was vortexed vigorously for 3 min followed by self-assembly under gentle stirring for 2 h at RT. The NPs were washed three times using an Amicon® Ultra-4 centrifugal filter with 30,000 Da MWCO (Millipore, Billerica, Mass.). The nanoparticles were resuspended in PBS buffer, pH 7.2, 2 mM EDTA and incubated with peptides (MW=1137.54 Da) at a 1/2 molar ratio to DSPE-PEG-Mal for 45 min at RT. The peptides were previously reduced using Bond-breaker TCEP solution, Neutral pH (Thermo Scientific, Rockford, Ill.) in PBS-EDTA at a 1/1 disulfide bond/TCEP molar ratio. Free peptides were removed using a Sephadex® G25 column. For scale-up, multiple vials of NPs were made with concentration and volume kept constant to maintain small NP diameters. TEM images of the nanoparticles were obtained using 1 mg/mL NPs stained with 3% uranyl acetate solution. Size (diameter, m) and surface charge (zeta potential, mV) were evaluated by quasi-elastic laser light scattering using a ZetaPALS dynamic light-scattering detector (15 mW laser, incident beam-676 nm; Brookhaven Instruments, Holtsville, N.Y.).

Results

To investigate the targeting properties of the candidate peptide against the basement membrane, a hybrid NP system was engineered to have a hydrophobic drug-eluting core, a hydrophilic polymeric shell, and a lipid monolayer, as described by Chan, et al. (2009) Biomaterials 30, 1627-1634. Poly(ethylene glycol) (PEG) covalently conjugated to 1,2-distearoyl-sn-glycero-3-phosphoethanolamine (DSPE) was used to form the hydrophilic polymeric shell. To complete the lipid monlayer, soybean lecithin, which is considered Generally Regarded as Safe (GRAS), was used to form the core-shell interface.

For the hydrophobic drug-eluting core, paclitaxel-polylactide (Ptxl-PLA) conjugates were synthesized by a drug/alkoxide-initiated ring-opening polymerization strategy as described in example 2. Ptxl was mixed with equimolar amounts of [(BDI)ZnN-(TMS)₂] (BDI=2-((2,6-diisopropylphenyl)amino)-4-(2,6-diisopropylphenyl)imino)-2-pentene, TMS=trimethylsilyl](9) and the (BDI)Zn-Ptxl complex formed in situ subsequently initiated and completed the polymerization of lactide within hours at room temperature (FIG. 2A). Ptxl was shown to be conjugated to the terminals of PLA by comparing the elution profile of free Ptxl to Ptxl-PLA by reverse phase-high performance liquid chromatography (RP-HPLC) (SI Methods). Ptxl-PLA eluted at approximately 21 min instead of the original approximately 14 min Ptxl peak (FIG. 2B).

In FIG. 2B, nanoparticle synthesis is illustrated in which the core (Ptxl-PLA conjugate) and shell (lipid and lipid-PEG) were integrated via nanoprecipitation and self-assembly. The KLWVLPK (SEQ ID NO:3) peptide was conjugated via a C-terminal GGGC (SEQ ID NO:28) linker to DSPE-PEG-maleimide using maleimide-thiol conjugation chemistry. Transmission electron microscopy (TEM) showed the spherical structures of the nanoparticles. The size and the surface zeta potential of non-functionalized NPs in water were 57.3±0.4 nm (mean±s.d.) and −12.83±2.73 mV (mean±s.d.). Peptide attachment resulted in an approximately 1 nm size increase and made the surface charge cationic, presumably because the peptides were N-terminus exposed to retain their original phage-displayed orientation.

To characterize the nanoparticles physiochemically, their release kinetics were quantified by taking aliquots at scheduled time points for RP-HPLC analysis. Ptxl was released by diffusion when the Ptxl-PLA ester bond was hydrolyzed. The amount of Ptxl released from Ptxl-LA₂₅ was 43.4% on day 2, and 91.0% and 93.8% on day 10 and day 12 respectively. In vitro drug release of Ptxl from the nanoparticle core is shown in FIG. 2C. Samples at different time points were measured for absorbance at 227 nm (mean±s.d., n=3).

Drug elution rates can be further controlled by varying lactide/Ptxl ratios during ring-opening polymerization, resulting in different PLA chain lengths attached to the Ptxl drug. The use of polymers to control Ptxl release is also a notable feature of drug eluting stents (DES), however, 80-90% of the Ptxl fraction is never released.

The two parameters of drug loading and release are important for drug efficacy. Increased drug loading into the particle core tends to reduce overall stability, giving an undesired burst release effect and reduced efficacy. Larger particles have slower release profiles, but when systemically administered are more readily detected and cleared from circulation, resulting in a lack of efficacy. For vascular targeting, since small particles show improved vessel adhesion and retention, incorporating slow-eluting conjugates into the nanoparticle design allows for: (i) improved drug encapsulation; (ii) sub-100 nm NPs for vascular targeting; and (iii) sustained drug release over two weeks.

### Example 3

### Cytotoxicity and Binding Studies

Materials and Methods

Human Aortic Smooth Muscle Cell (haSMC) Cytotoxicity Studies

96-well plates were Matrigel-coated and BSA-blocked in PBS. HaSMC were plated at 10,000 cells/well in a 37° C./5% CO₂ incubator and grown for 24 h in Medium 231 supplemented with 10 μg/mL gentamycin, 0.25 μg/mL amphotericin B, and smooth muscle growth supplement (all from Cascade Biologics, Invitrogen). Treatment groups (n=5) included nanoparticles, scrambled-peptide NPs, non-targeted NPs, four-fold dilutions of Ptxl (in maximum 0.1% DMSO) in media and a media-only control. Samples were incubated with cells for 45 min. The wells were washed two times with complete media and replaced with fresh complete media for 48 h. Medium 231 was replaced with phenol red-free RPMI medium supplemented with 10% fetal bovine serum (Invitrogen) containing [3-(4,5-dimethylthiazol-2-yl)-5-(3-carboxymethoxyphenyl)-2-(4-sulfophenyl)-2H-tetrazolium, inner salt] (MTS) and phenazine methosulfate (PMS) and incubated for 2 h at 37° C. (CellTiter 96° AQᵤₑₒᵤₛ Non-Radioactive Cell Proliferation Assay, Promega, Madison, Wis.). Formazan product absorbance was measured at 490 nm against a reference wavelength of 650 nm.

Balloon-Angioplasty Ex Vivo and In Vivo Studies

Sprague Dawley rats weighing approximately 450-500 g were obtained from Charles River Laboratories (Wilmington, Mass.) and fed a normal rodent diet. All animal procedures were conducted by a certified contract research organization using protocols consistent with local, state and federal regulations as applicable and approved by the Institutional Animal Care and Use Committee (IACUC).

For ex viva studies, animals were sacrificed for open abdominal cavity surgery in situ. Aortas were flushed with saline and injured by four passages of a Fogarty® arterial embolectomy 2F balloon catheter (Model: 120602F, Edwards Lifesciences, Irvine, Calif.) in a rotating fashion. AlexaFluor 647 (A647) dyes were covalently conjugated to PLGA (viscosity 0.19 dl/g) using EDC/NHS chemistry in DMF. The A647-PLGA conjugates were precipitated in 2/1 ethyl ether/methanol by centrifugation, dried in vacuum and resuspended in acetone for NP preparation. Fluorescence (relative units) was quantified using the GeminiXPS Microplate Spectrofluorometer (Molecular Devices) and samples were diluted accordingly in PBS for comparable delivery of fluorescence into the aortas. 0.4 mL samples (approximately 6 mg/mL) were incubated in the aorta for 5 min using metal clips to secure both ends of the aorta. Non-adsorbed samples were flushed away with saline using an Advance Infusion Pump Series 1200 syringe pump (Roboz Surgical Instrument Co., Gaithersburg, Md.) programmed at 4 mL/min for 15 min.

For in vivo intraarterial (IA) studies, animals were anesthesized intramuscularly with ketamine (60 mg/kg)/kylzaine (10 mg/kg) and given buprenorphine as an analgesic. Left common carotids were injured by four passages of the 2F balloon-catheter, before a 30-gauge tubing was introduced via the external carotid into the common carotid and advanced beyond the angioplastied region into the aortic arch. Samples (approximately 10 mg/mL) were infused at 1 mL/min for 1 min. The external carotids were permanently ligated. Animals were sacrificed 1 h after surgery and the carotids were harvested.

For in vivo intravenous (IV) studies, animals were additionally given heparin (500 IU/kg) by IV injection immediately before surgery. Animals were subject to left common carotid artery surgery and samples (approximately 15 mg/mL) were given by a 1 mL IV tail vein injection. Animals were sacrificed after 1 h and the carotids were harvested.

Matrigel Binding Studies

96-well plates were coated with 100 μL 1/50 dilutions of Matrigel in TBS overnight at 4° C., or TBS buffer only. Plates were blocked with 3% BSA/TBS for 2 h at RT and washed three times. 10¹⁰ p.f.u. of each phage clone was added in 0.5% TBST in triplicate to either Matrigel or BSA-coated wells. Bound phage particles were detected with peroxidase-conjugated mouse anti-M13 monoclonal antibodies at 1/5000 dilution (Amersham Pharmacia Biotech, Piscataway, N.J.). After a 1 h incubation, the reaction was developed with 2,2′-azino-bis(3-ethylbenzthiazoline-6-sulphonic acid) (ABTS) (Amersham) and the absorbance was read at 405 nm against a reference wavelength of 490 nm with a SpectraMax Plus 384 Microplate reader (Molecular Devices, Sunnyvale, Calif.).

IC₅₀ Value Determination of Phage Clones

Matrigel-coated and blocked 96-well plates were incubated in triplicate with 10⁻⁵-10⁻¹⁰ M peptide concentrations and 10⁹ pfu of phage in 100 μL 0.5% TEST. After 1 h at RT, bound phages were labeled with anti-M13 mAb and color was developed and detected using ABTS absorbance (405-490 nm). Peptide inhibition curves were normalized on a percentage scale to provide an estimate of the IC₅₀, which was calculated using a dose-response curve fit using the Origin 8 data analysis software (Northampton, Mass.) by the formula:

Y=Bottom+(Top−Bottom)/(1+10̂((Log IC50−X)*HillSlope)).

Release Kinetics Studies

To assess the Ptxl-PLA bond, Ptxl and Ptxl-PLA conjugates were subject to quantitative analysis using an Agilent 1100 HPLC (Paolo Alto, Calif.) equipped with a pentafluorophenyl column (Curosil-PFP, 250×4.6 mm, 5 μm; Phenomenex, Torrance, Calif.). Ptxl and Ptxl-PLA absorbance was measured by an UV-Vis detector at 227 nm in a 1/1 acetonitrile/1% trifluoroacetic acid 1 mL/min non-gradient mobile phase. To quantify the Ptxl-PLA drug release profile, a 2 mg nanoparticle sample was divided equally into Slide-A-Lyzer® MINI dialysis microtubes with a molecular weight cut-off of 20,000 Da (Pierce, Rockford, Ill.). Experiments were carried out in triplicate in PBS at 37° C. Remaining Ptxl-PLA was quantified by absorbance at 227 nm using reverse phase-HPLC.

Optical 3D Imaging and Fluorescence Microscopy Studies

All tissues were fixed in 4% paraformaldehyde/4% sucrose/saline overnight at 4° C. Whole tissue sections were imaged simultaneously using the IVIS Imaging System 200 Series (Hopkinton, Mass.) at 640/700 (ex/em) wavelength, exposure time=1 s, binning=medium, F/Stop=2. Tissue sections were overlayed onto photographs taken at binning=medium, F/Stop=8. After IVIS imaging, the same tissues were OCT-frozen and cut to give approximately 10 μM sections for fluorescent microscopy. Representative H&E slides were done on paraffin-fixed sections. All histology sections were done by the Massachusetts Institute of Technology Koch Institute Histology Facility and imaged using a Delta Vision RT Deconvolution Microscope using the 20× objective (Applied Precision Ine, Issaquah, D.C.).

Statistical Analysis

Student's t-test or one-way ANOVA with post-hoc Tukey tests were used to determine significance. All error bars represent the standard deviation of the mean.

Results

To validate the therapeutic efficacy of this treatment, a human aortic smooth muscle cell (haSMC) cytotoxicity study was used to assess nanoparticle differential cellular cytotoxicity and binding affinity on Matrigel-coated wells with haSMC (FIG. 3). FIG. 3 is a graph showing human aortic smooth muscle cell (haSMC) cytotoxicity studies as a function of binding affinity. HaSMC on Matrigel-coated plates were incubated with nanoparticles (T); scrambled-NPs (S); or non-targeted bare-NPs (B); four-fold dilutions of Ptxl without nanoparticles; and a media-only control for 45 min.

To test the sequence specificity of the KLWVLPK (SEQ ID NO:3) peptide (T), two controls: scrambled PWKKLLV (SEQ ID NO:26) peptide (S) and non-targeted (B) NPs, were designated. In addition, a media-only control and four-fold dilutions of free Ptxl in DMSO (maximum 0.1% DMSO in media) were tested. The maximum free Ptxl concentration used was 51 μM, exceeding by two log scales a suitable Ptxl dose range of 50-1000 nM for haSMC cytotoxicity. An incubation time of 45 min was significantly shorter than the typical incubation time of Ptxl (approximately 4-24 h). The wells were rinsed twice with complete media and further incubated with fresh media for 48 h. Unlike free Ptxl which is removed during the washing step, the nanoparticles attached to the collagen IV matrix were retained for continued Ptxl release. Hence, the nanoparticles demonstrated the greatest cellular cytotoxicity (n=5, p<0.001) on haSMCs as a function of increased binding affinity on Matrigel-coated plates.

Binding Studies in Angioplasty Models of Injured Vasculature.

The targeting affinity of the nanoparticle system towards injured vasculature was evaluated. To create those vascular characteristics, a Fogarty 2-French balloon catheter was used to injure rat arteries by repeatedly advancing, inflating the balloon and withdrawing to denude the endothelial monolayer and expose the basement membrane. This loosely mimics a percutaneous angioplasty procedure in human patients, the difference being that in human patients the catheter is inflated locally in a pre-existing stenotic lesion. A representative H&E stained cross-section shows an injured aorta with the endothelial layer removed, and an uninjured aorta with an intact endothelial monolayer.

Balloon-injury removes the endothelial cell (EC) monolayer. Ex vivo delivery was tested in an abdominal aorta injury model. Samples were delivered into the aorta segment for 5 min in situ. Non-adsorbed samples were flushed out by saline infusion for 15 min. Fluorescence images were overlayed on photographs of balloon-injured aortas incubated with nanoparticles, compared with scrambled-peptide and non-targeted NPs. In vivo intraarterial delivery was also tested in a carotid injury model. A catheter was introduced via the external carotid into the common carotid and advanced into the aortic arch. Samples were delivered at 1 mL/min for 1 min and allowed to circulate for 1 h before the animals were sacrificed. Fluorescence images were overlayed on photographs of carotid arteries incubated with the nanoparticles, compared with scrambled-peptide and non-targeted NPs. In vivo systemic delivery was tested in a carotid angioplasty model. Samples were delivered by 1 mL intravenous tail vein injection and allowed to circulate for 1 h before the animals were sacrificed. Fluorescence images were overlayed on photographs of carotid arteries incubated with the nanoparticles, compared with scrambled-peptide and non-targeted NPs. For imaging, Alexa Fluor 647-PLGA dye conjugates were encapsulated in place of Ptxl-PLA drug conjugates.

The ex vivo study examined targeting of the nanoparticle system to balloon-injured rat aortas. Alexa Fluor 647 fluorescent dye-poly(lactic-co-glycolic acid) (A647-PLGA) conjugates were substituted for Ptxl-PLA drug conjugates to visualize the nanoparticles by fluorescence microscopy and optical 3D imaging. This wavelength is beyond the autofluorescence range of typical endogenous tissue fluorophores such as collagen and elastin which excite and emit maximally at approximately 300-500 nm. Therefore, any A647-PLGA fluorescence detected would be NP deposition. A647-PLGA encapsulated nanoparticles were incubated in the abdominal aorta for 5 min under constant pressure, followed by extensive washing using a syringe-pump to remove non-adsorbed samples. Subsequently, the abdominal aortas were harvested and viewed by whole vessel fluorescent 3D optical imaging. Fluorescence quantification using the region-of-interest (ROI) function allowed quantification of nanoparticle retention, shown in FIG. 5, measurements are average fluorescent efficiency per unit area (cm⁻²). Efficiency measurements are independent of the lumination intensity, and the value of each pixel represents the fractional ratio of emitted photons per incident excitation photon. The nanoparticles bound to balloon-injured aortas at 1.43±0.48×10⁴ cm⁻², while scrambed-peptide and non-targeted NPs overall bound two-fold less at 48% (n=3, p<0.05) and 47% (n=3, p<0.05) respectively. To ensure that the nanoparticles would not target intact endothelial layers, they were also incubated with uninjured aortas and bound four-fold less at 3.39±0.50×10⁻⁵ cm⁻² (n=3, p<0.01) compared to injured vessels. Frozen histological sections were photographed to show the distribution of the nanoparticles along the arterial cross-section.

Samples were delivered into the aorta segment for 5 min in situ and non-adsorbed samples were flushed out by saline infusion for 15 min. A catheter was introduced via the external carotid into the common carotid and advanced into the aortic arch. Samples were delivered at 1 mL/min for 1 min and allowed to circulate for 1 h before the animals were sacrificed. Samples were delivered by 1 mL intravenous tail vein injection and allowed to circulate for 1 h before the animals were sacrificed. For imaging, Alexa Fluor 647-PLGA dye conjugates were encapsulated in place of Ptxl-PLA drug conjugates. The scale bar in all images is 1 cm.

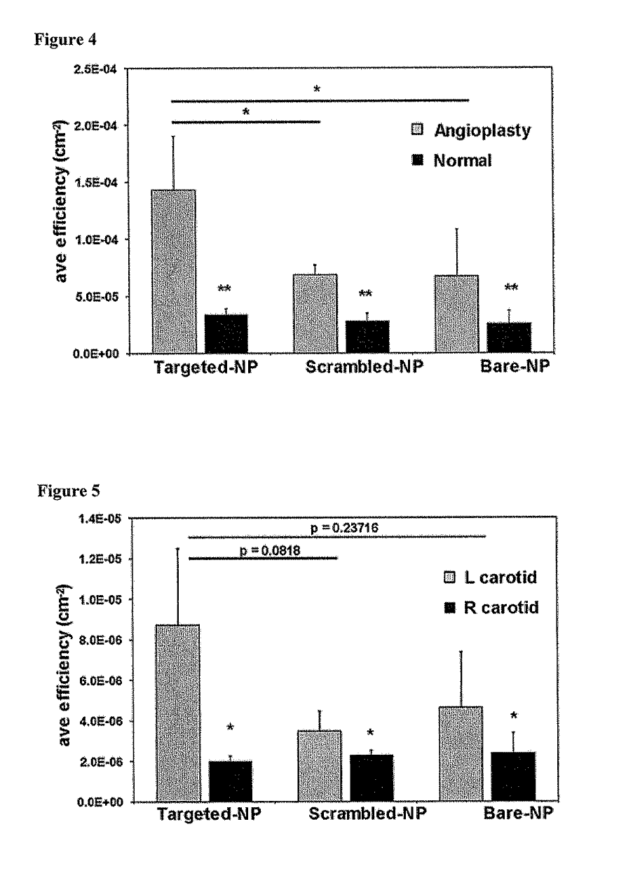

FIG. 4 is a graph of the quantification of nanoparticle binding ex vivo to angioplastied aortas, Aorta sections (n=3) were analyzed using the region-of-interest (ROI) function of the IVIS Living Image Software and shown as average efficiency values per unit area (cm⁻²) of mean±s.d. (*) P<0.05, (**) P<0.01 by one-way analysis of variance with Tukey post-hoc test.

Targeting in vivo via intraarterial (IA) infusion was examined using the left carotid injury model. The nanoparticles were injected into angioplastied left carotids through a catheter positioned in the aortic arch inserted from the external carotid artery over the course of 1 min, and allowed to circulate for 1 h. More nanoparticles (8.71±0.38×10⁻⁶ cm⁻²) were found in the angioplastied left carotid artery compared to the right carotid by four-fold; while the scrambled-peptide and non-targeted NPs were retained in the left carotids at 40% (n=5, p=0.0818) and 53% (n=5, p=0.23716) of nanoparticles respectively (FIG. 5).

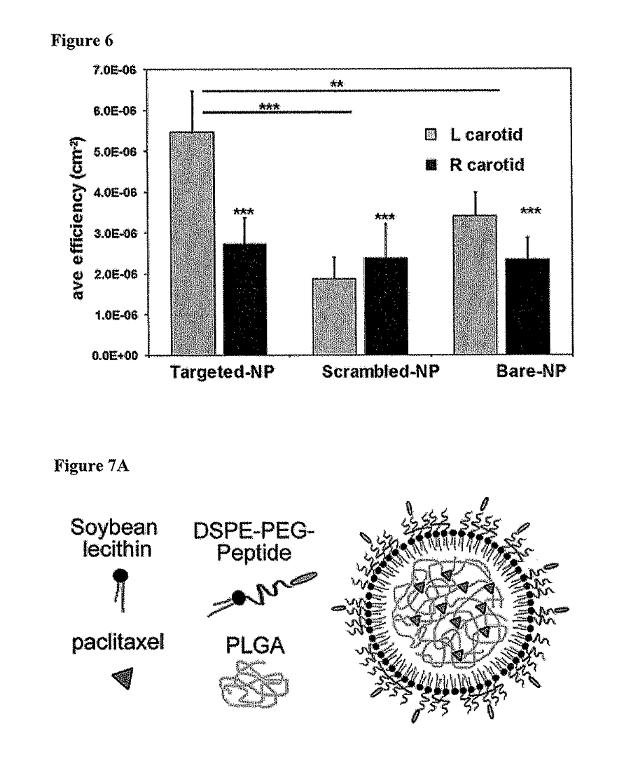

FIG. 6 is a graph of the quantification of nanoparticle binding in vivo to angioplastied left common carotids by intraarterial delivery. Both the left and right common carotid arteries (n=3) were analyzed using the region-of-interest (ROI) function of the IVIS Living Image Software and shown here as average efficiency per unit area (cm⁻²) of mean±s.d. (*) P<0.05 by one-way analysis of variance with Tukey post-hoc test.

The nanoparticle system was studied for systemic delivery because repeat dosing may be helpful in the treatment of chronic vascular disease. Using the left carotid injury model, the nanoparticles were given as a 1 mL intravenous (IV) dose via tail-vein injection and allowed to circulate for 1 h Nanoparticle retention was 5.46±1.02×10⁻⁶ cm⁻² in the angioplastied left carotids compared to scrambled-peptide and non-targeted NPs, which were 35% (n=5, p<0.001) and 64% (n=5, p<0.01) of nanoparticles respectively. The nanoparticles bound to the left carotids two-fold over the right healthy carotids (p<0.001) are shown in FIG. 6.

FIG. 6 is a graph of the quantification of nanoparticle binding in vivo to angioplastied left common carotids by intravenous delivery. Both the left and right common carotid arteries (n=5) were analyzed using the region-of-interest (ROI) function of the IVIS Living Image Software and shown here as average efficiency per unit area (cm⁻²) of mean±s.d.

The binding studies to injured vasculature show the successful targeting and retention of nanoparticles to injured carotid arteries in vivo and abdominal aortas ex vivo.

### Example 4

### Synthesis and Characterization of NP Treatment Groups

Materials and Methods

Materials

Peptide sequences (KLWVLPKGGGC-Am, SEQ ID NO:27) were custom synthesized by Genscript (Piscataway, N.J.) and purified by RP-HPLC to ≧0.95 by mass spectral analysis (MW: 1157.43 Da). Soybean lecithin containing 0.9-0.95 soybean phosphatidylcholine by mass was obtained from MP Biomedicals (Solon, Ohio). DSPE-PEG2000 and DSPE-PEG2000-maleimide were obtained from Avanti (Alabaster, Ala.). PLGA with 1/1 lactide/glycolide monomer ratio, ester-terminated and 7.2-9.2 L/g inherent viscosity was purchased from Durect Corporation (Pelham, Ala.). ¹⁴C-paclitaxel (benzoyloxy ring-¹⁴C, 50-100 mCi/mmol) in ethyl acetate solution (100 μCi/mL) was purchased from Moravek Biochemicals Inc. (Brea, Calif.). ³H-PLGA in ethyl acetate solution (5 mCi/mL) was custom synthesized by PerkinElmer (Waltham, Mass.). Paclitaxel and other materials were purchased from Sigma-Aldrich unless otherwise noted.

Synthesis of Targeted Lipid-Polymeric Nanoparticles

The targeted lipid-polymeric nanoparticles were synthesized as described by Chan J M, et al. Proc Natl Acad Sci USA. 2010; 107(5):2213-2218, using pre-conjugated DSPE-PEG-peptide triblock ligands. Briefly, peptides were reduced for 30 min using Bond-breaker TCEP solution, Neutral pH (Thermo Scientific) in pH 7.2 PBS buffer with 5 mmol/L EDTA at a 1/1 disulfide bond/TCEP molar ratio. DSPE-PEG-peptide triblocks were synthesized in 1/25 Ethanol/H₂O (lipid solvent) at a peptide/DSPE-PEG 5/4 molar ratio for 4 h with gentle rocking at room temperature (RT). Free peptides were removed by dialysis in 3,500 Da molecular weight cut-off (MWCO) membranes (Spectrum Laboratories, Houston, Tex.) overnight with two water changes. 1.5 mg DSPE-PEG-peptide/DSPE-PEG (1/9 molar ratio) and 0.75 mg lecithin in the lipid solvent was heated to 68° C. for 3 min under gentle stirring. 9 mg of PLGA and 0.45 mg of paclitaxel in 3 mL acetone (5/100 paclitaxel/PLGA by mass) was added dropwise at 3 mL/min. The mixture was vortexed vigorously for 3 min and stirred gently over 2 h at RT. The targeted lipid-polymeric nanoparticles were washed three times with a 30,000 Da MWCO Amicon Ultra-4 centrifugal filter (Millipore, Billerica, Mass.). The targeted lipid-polymeric nanoparticles were dissolved in saline (9 g/L NaCl in H₂O) and filtered using 0.8 μm Supor membrane syringe filters (Pall Corporation, Port Washington, N.Y.). Concentrations and volumes of the reaction were maintained during scale-up.

Transmission Electron Microscopy (TEM) Characterization

TEM experiments were performed using the JEOL JEM-200CX at an acceleration voltage of 200 kV. TEM grids were prepared by adding NP samples (2 mg/mL) in H₂O onto 300-mesh Formvar-coated copper grids (Electron Microscopy Sciences, Hatfield, Pa.). Samples were blotted away after 10 min and the grids were negatively stained for 10 min at RT with freshly prepared, sterile-filtered 30 g/L uranyl acetate solution. The uranyl acetate solution was blotted away and the grids were air dried prior to imaging.

HPLC Quantification of Paclitaxel Loading and Release Kinetics

The drug loading in all paclitaxel and NP batches were quantified by RP-HPLC against a standard curve of known paclitaxel concentration before i.v. injection. All discussions of drug dosing in units of mg/kg relate to the active drug composition. To measure the drug loading yield and release profile of paclitaxel from each type of NP, 3 mL NP solutions at a concentration of 0.5 mg/mL were split equally into 33 Slide-A-Lyzer MINI dialysis microtubes, 10,000 Da MWCO (Pierce, Rockford, Ill.) and dialyzed against 3.5 L PBS at 37° C. PBS was changed periodically during the dialysis process. At the indicated times, the total solution in each microtube was recorded (n=3) and 0.1 mL of the solution per tube was mixed with an equal volume of acetonitrile to dissolve the NPs. Paclitaxel content was quantified by RP-HPLC using an Agilent 1100 HPLC (Paolo Alto, Calif.) equipped with a pentafluorophenyl column (Curosil-PFP, 250×4.6 mm, 5 μm; Phenomenex, Torrance, Calif.). Paclitaxel absorbance was measured at 227 nm using a UV-Vis detector with a retention time of ˜12-14 min in a 1 mL/min, 1/1 acetonitrile/water, non-gradient mobile phase. Finally, the amount of paclitaxel retained was calculated based on the original volume collected in the microtube.

Pharmacokinetic Studies of Targeted Lipid-Polymeric Nanoparticles

12 male Sprague Dawley rats weighing approximately 250 g with bilateral jugular catheters were obtained from Charles River Laboratories (Wilmington, Mass.). To assess paclitaxel levels in circulation, animals were injected with 400 μL of ¹⁴C-paclitaxel-encapsulated targeted lipid-polymeric nanoparticles in saline (5 μCi/mL) via the left catheter. 200-500 μL of blood was collected via the right catheter at 1, 3, 6, 9 and 24 h after injection (n=6). In a separate study to assess targeted lipid-polymeric nanoparticle polymer levels in circulation, 400 μl, of NPs synthesized with ³H-PLGA was injected in saline (100 μCi/mL) and 100 μL of blood was collected at 1, 3, 6, 9, 24, 48, 96 and 120 h after injection (n=6). Blood radioactivity was quantified as disintegrations per minute (DPM) using the PerkinElmer Tri-Carb 2810TR liquid scintillation analyzer.

Blood and tissue samples were processed as described by Chan J M, et al. Methods Mol. Biol. 2010; 624:163-175, with modifications. Solvable tissue solubilizer and Hionic-Fluor scintillation cocktail were purchased from PerkinElmer (Waltham, Mass.). All samples were weighed before processing. To prepare blood samples for radioisotope counting, 1.5 mL of tissue solubilizer was added to ≦0.5 mL blood and incubated at 55-60° C. for 2 h to dissolve the blood samples. Next, 50 μL of 0.5 mol/L EDTA-di-sodium salt solution was added to reduce foaming and 0.5 mL of hydrogen peroxide (300 g/L) was added dropwise with gentle agitation. Samples were further incubated at 55-60° C. for 1 h. To prepare tissue samples for radioisotope counting, kidney, spleen, liver, heart, and lung tissues (≦150 mg) were similarly processed, except with 4.0 mL of tissue solubilizer instead. Finally, 16 mL of Hionic-Fluor scintillation cocktail was added to both blood and tissue samples, and samples were subjected to temperature and light-adaptation for 1 h before counting. Sample radioactivity was read as disintegrations per minute (DPM) of either ¹⁴C or ³H radioisotopes using the PerkinElmer Tri-Carb 2810TR liquid scintillation analyzer, and standardized as DPM per gram of tissue or blood (DPM/g) based on the weight of the sample.

Results

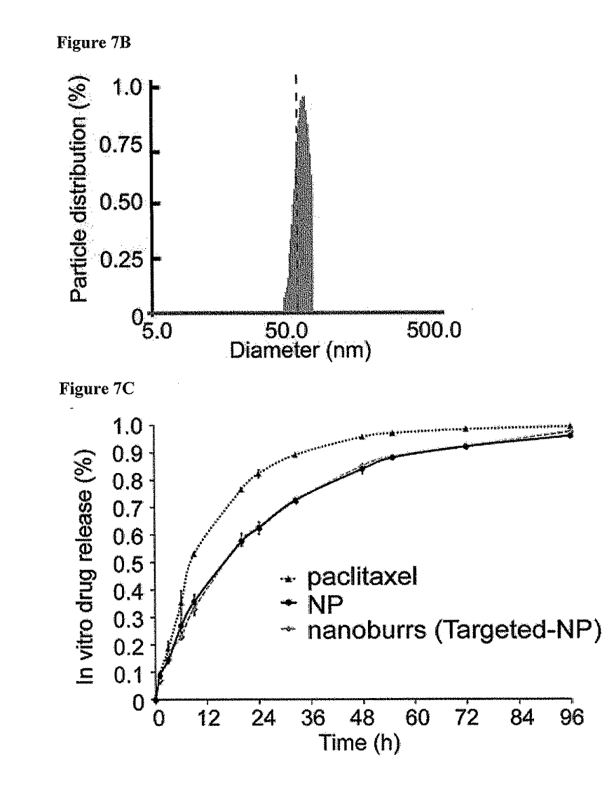

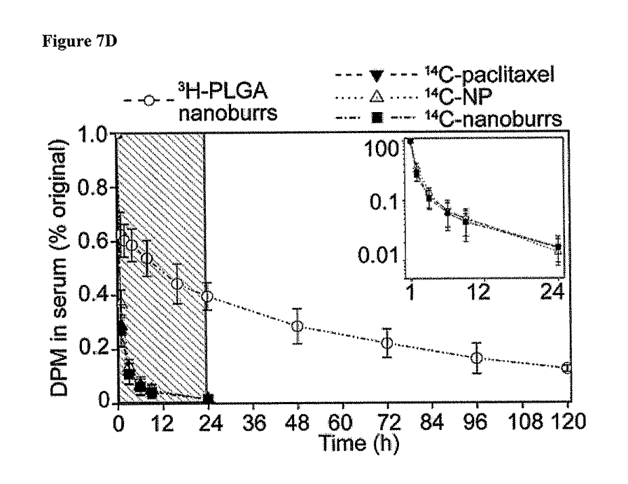

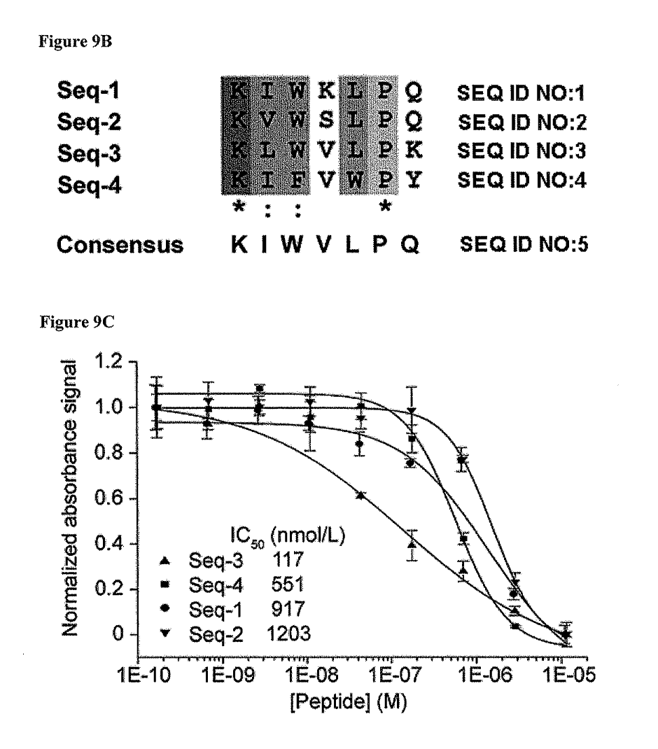

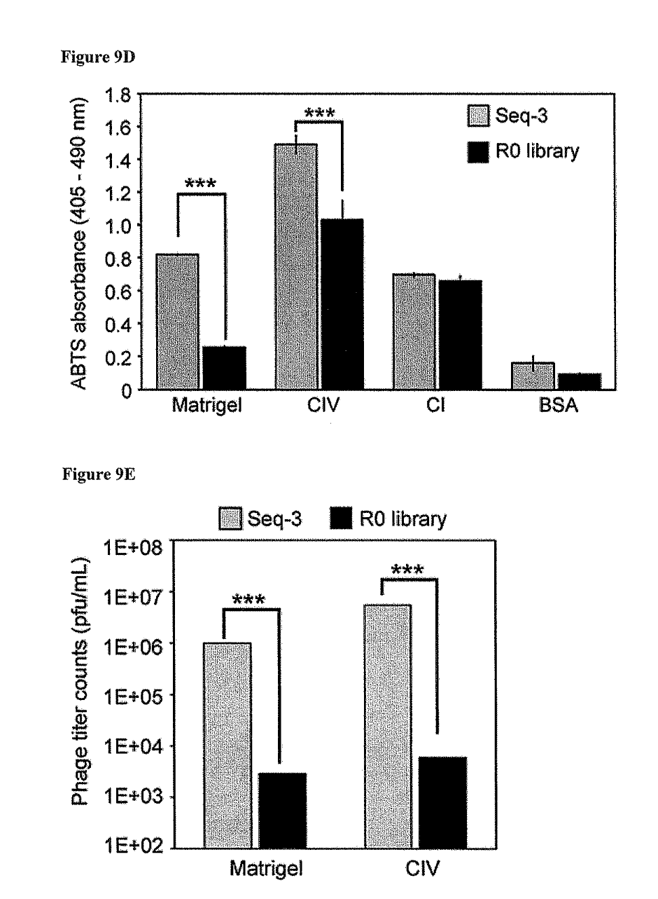

Targeted lipid-polymeric nanoparticles with a core-shell lipid-polymeric structure were formulated by nanoprecipitation and self-assembly (Chan J M, et al. Proc Natl Acad Sci USA. 2010; 107:2213-2218) (FIG. 7A). Determination of a specific collagen IV binding site by introducing point mutations is inherently difficult since collagen IV uses glycines (core) and prolines (bends) to form its triple helical structure. Post-translational modifications to the dominant glycine-proline-hydroxyproline motif may not be genetically altered either. Given these limitations, peptide affinity was characterized by site-specific binding competition of phage-displayed peptides against synthetic peptides on Matrigel, a basement membrane extract rich in collagen IV, and showed dose-response competition at IC₅₀=114 nmol/L (Chan J M, et al. Proc Natl Acad Sci USA. 2010; 107:2213-2218) (FIG. 9). Other analyses included Clustal alignment, PyMol visualization, and phage titer analyses against collagen IV (FIG. 9). Peptides were synthesized with a linker sequence (GGGC, SEQ ID NO:28) at the C-terminus for thiomaleimide coupling. NP sizes measured with dynamic light scattering were found to be 55.1±0.4 nm (polydispersity=0.075, n=3) in one representative batch after filtration, with batch-to-batch variation under ±5 nm (FIG. 7B). NP batches functionalized with peptides did not show a significant size increase beyond 5 nm. TEM images obtained with 2 mg/mL targeted lipid-polymeric nanoparticles stained with uranyl acetate solution showed that the NPs were spherical, monodisperse and in the 50 nm size range (FIG. 7B).

To determine the final drug loading based on a 5/100 paclitaxel/PLGA input, NP batches (n=3) were lyophilized to obtain the final PLGA polymer weight (0.8 of final mass; as the other 0.2 is lipid and PEG mass). Drug content was measured by RP-HPLC, with the encapsulation efficiency calculated to be ⅕ of the drug input weight, and the final drug load was determined to be 1/100 paclitaxel/PLGA polymer weight. In comparison with previous studies where drug burst release was observed with higher drug loading (Zhang L, et al. ACS Nano. 2008; 2:1696-1702), a burst release was not observed at this paclitaxel load (FIG. 7C).

To measure drug release rates in vitro, NP samples were dialyzed in 3.5 L of PBS buffer at 37° C. and samples were withdrawn at indicated time points. The drug release half-life was determined to be approximately 17.77 h for the targeted lipid-polymeric nanoparticles and 18.24 h for the NP groups, which suggests that peptide conjugation only slightly interfered with the self-assembly process, and marginally increased rates of drug release. For paclitaxel in solution, the drug release half-life was found to be approximately 10.5 h (FIG. 7C).

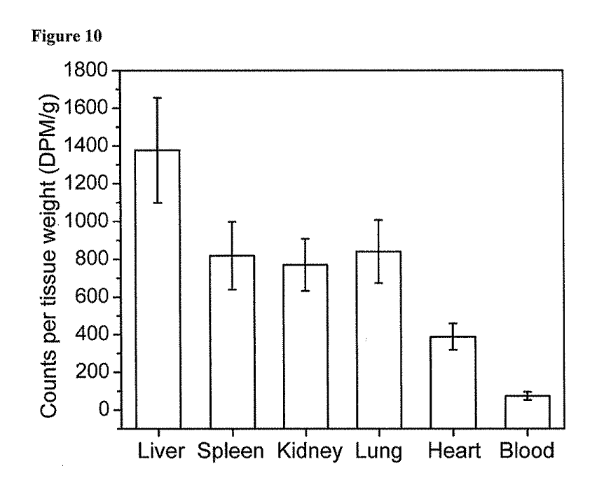

To investigate the pharmacokinetic properties of the targeted lipid-polymeric nanoparticles, serum half-life studies were carried out to measure paclitaxel and polymer (PLGA) levels over time in vivo (FIG. 7D). ¹⁴C-paclitaxel-encapsulated paclitaxel, NP, and targeted lipid-polymeric nanoparticle samples, and ³H-PLGA-encapsulated targeted lipid-polymeric nanoparticles were delivered intravenously and blood samples were collected to quantify blood concentrations of paclitaxel and PLGA, respectively. The pharmacokinetic data obtained was fitted using a biexponential model for distribution phase half-life (t_{1/2α}) and terminal half-life (t_{1/2β}) (Table 1). The biodistribution of ¹⁴C-paclitaxel delivered by the targeted lipid-polymeric nanoparticles in the liver, spleen, kidney, heart and lungs was determined at 24 h (FIG. 10).

TABLE 1
Pharmacokinetic parameters of treatments administered
with ¹⁴C-paclitaxel or ³H-PLGA

| Radioisotope conjugate | Treatment group | t_{1/2α} (h) | t_{1/2β} (h) |
|---|---|---|---|
| ¹⁴C-paclitaxel | paclitaxel (n = 6) | 0.64 | 8.02 |
| | NP (n = 6) | 0.49 | 9.78 |
| | targeted lipid- | 0.51 | 8.84 |
| | polymeric | | |
| | nanoparticles (n = 6) | | |
| ³H-PLGA | targeted lipid- | 1.48 | 34.64 |
| | polymeric | | |
| | nanoparticles (n = 6) | | |

t_{1/2α} (h), half-life of distribution phase;
t_{1/2β} (h), half-life of elimination phase

Pharmacokinetic parameters of paclitaxel, NP, and targeted lipid-polymeric nanoparticle treatment groups were determined by a bi-exponential model which characterizes the kinetics of tissue distribution and elimination from the plasma compartment as two exponential phases (Fetterly G J, et al. AAPS PharmSci. 2003; 5(4):E32). Both radioisotope calculations were subject to background subtraction, factoring in baseline proton-exchange with ³H-PLGA (Waterfield W R, et al. Nature. 1968; 218(5140):472-3).

When paclitaxel was tracked, the plasma concentration of ¹⁴C-paclitaxel decreased bi-exponentially after the bolus intravenous injection, with distribution phase (t_{1/2α}) of the paclitaxel group (0.64 h) longer than NP (0.49 h) and targeted lipid-polymeric nanoparticle (0.51 h) treatment groups; but with a terminal half-life (t_{1/2β}) in the paclitaxel group (8.02 h) that is shorter than the NP (9.78 h) and targeted lipid-polymeric nanoparticles (8.84 h) treatment groups. When the polymer was tracked only for the targeted lipid-polymeric nanoparticle group, the plasma concentration of ³H-PLGA decreased bi-exponentially after bolus intravenous injection, with a distribution phase of 1.48 h and a terminal half-life of 34.64 h. NP complete clearance with clinical significance occurred by 120 h.

### Example 5

### Tolerability Studies

Materials and Methods

Formulation Tolerability Studies

54 male Swiss albino mice weighing approximately 25-30 g were obtained from Charles River Laboratories, A single preparation was used for each formulation, i.e., 2.5 mg/mL targeted lipid-polymeric nanoparticles and 1.2 mg/mL paclitaxel based on the active drug composition. Paclitaxel in solution was prepared as described by Gelderblom H, et al. Clin Cancer Res. 2002; 8:1237-1241, in a 1:1 volume ratio of Cremophor EL and USP-grade ethanol, diluted in saline to 0.3-1.2 mg/mL and sterile-filtered. Animals were given a single bolus i.v. dose via the tail vein (n=4 per group).

Clinical monitoring over seven days was carried out for any signs of adverse effects. Tolerated doses of the treatment were defined as follows: (a) No lethal toxicity in treated mice; (b) Daily monitoring of mice body weight produced an animal body weight loss of <0.1 of the original weight before treatment; (c) No neurotoxicity as defined as the appearance of neuromuscular symptoms such as tremors, ataxia, or paraplegia; and (d) Regular blood hematology and biochemical parameters.

At the end of the study, animals were sacrificed by CO₂ inhalation. Gross necropsy was performed and H&E sections of the major organs (liver, lung, heart, spleen, kidney, nerves) were examined for any signs of toxicity. In a parallel study, blood samples from saline, 10 mg/kg paclitaxel and 35 mg/kg targeted lipid-polymeric nanoparticle treatment groups (n=6 per group) were taken at Day 7 for hematological analysis and assessment of biochemical parameters.

Results

Maximum tolerated dose (MTD) studies were carried out in healthy Swiss albino mice comparing targeted lipid-polymeric nanoparticles to paclitaxel using a single-dose i.v. injection. The Food and Drug Administration (FDA)-approved paclitaxel formulation uses Cremophor-EL as a solubilizing agent. This formulation has been shown to cause neuropathy, complement activation and hypersensitivity reactions which necessitate steroid pre-medication (Gelderblom H, et al. Eur J Cancer. 2001; 37:1590-1598). The paclitaxel MTD value in experimental animals varies in different studies (Hureaux J, et al. Pharm Res. 27:421-430; Kim S C, et al. J Control Release. 2001; 72:191-202). In this study, the paclitaxel MTD was found to be 10 mg/kg in mice, consistent with previous reports. 15 mg/kg paclitaxel doses caused the immediate death of two mice, possibly related to inadequate blood solubility at 1.2 mg/mL doses. In contrast, targeted lipid-polymeric nanoparticles dosed at 35 mg paclitaxel/kg in mice (2.5 mg/mL concentrations in saline) were well tolerated, suggesting an advantage from improved drug solubility and NP compatibility. Higher doses were not given to avoid exceeding the maximum volume that can be safely injected as a bolus (approximately 10-15 mL/kg body weight). Daily clinical observations were performed to monitor for adverse medical, cognitive or behavioral effects. The animals were weighed daily and monitored for hair loss, vomiting or diarrhea. The animals were also monitored for signs of tremors, staggering, drowsiness and general responsiveness.

At 10 mg/kg paclitaxel and 35 mg/kg targeted lipid-polymeric nanoparticles, neither regimen caused adverse cognitive or behavioral effects, while very marginal body weight loss occurred (<0.1 of initial mass). Blood samples were also collected at day 7 for hematological analysis. The readings taken for the 10 mg/kg paclitaxel group indicated signs of mild thrombocytopenia (platelet count<400×10⁹/L) in two mice, but otherwise readings were within the expected range (Table 2). Neither regimen had significant effects on various biochemical parameters suggestive of hepatic and renal injury (Table 2). Upon study termination, gross necropsies were performed and histological cross-sections were analyzed by a pathologist, with no significant findings noted.

TABLE 2
Hematological analysis and serum biochemical parameters from Swiss
Albino mice 168 h after a single intravenous dose.

| | | | targeted lipid- |
|---|---|---|---|
| | | | polymeric |
| | | paclitaxel, | nanoparticles, |
| | Saline | 10 mg/kg | 35 mg/kg |
| RBC, ×10¹²/L | 8.09 ± 0.676 | 8.30 ± 0.598 | 7.94 ± 0.512 |
| HGB, | 8.50 ± 0.69 | 8.81 ± 0.49 | 8.38 ± 0.43 |
| mmol/L | | | |
| HCT, % | 0.49 ± 0.03 | 0.493 ± 0.03 | 0.469 ± 0.02 |
| MCV, fL | 60.6 ± 1.95 | 59.5 ± 1.55 | 59.2 ± 2.51 |
| MCH, pg | 16.8 ± 0.386 | 17.1 ± 0.679 | 17.0 ± 0.629 |
| MCHC, | 17.3 ± 0.37 | 17.9 ± 0.47 | 17.8 ± 0.547 |
| mmol/L | | | |
| PLT, ×10⁹/L | 1 395 ± 42.1 | 1026 ± 533 | 1415 ± 514 |
| WBC, ×10⁹/L | 2.03 ± 0.677 | 2.05 ± 0.454 | 3.79 ± 0.741 |
| NEUT, % | 0.003 ± 0.002 | 0.002 ± 0.002 | 0.003 ± 0.002 |
| LYMPH, % | 0.87 ± 0.17 | 0.69 ± 0.13 | 0.77 ± 0.16 |
| Mono, % | 0.037 ± 0.05 | 0.167 ± 0.064 | 0.114 ± 0.072 |
| Eo, % | 0.0019 ± 0.002 | 0.0023 ± 0.004 | 0.0012 ± 0.002 |
| Baso, % | 0.090 ± 0.125 | 0.140 ± 0.078 | 0.108 ± 0.089 |
| ALP, U/L | 39.8 ± 3.24 | 41.7 ± 1.77 | 43.6 ± 2.73 |
| AST, U/L | 109.50 ± 11.22 | 119.83 ± 8.23 | 114.67 ± 12.51 |
| ALT, U/L | 65.23 ± 10.40 | 61.01 ± 6.10 | 66.10 ± 4.47 |
| BUN, | 8.40 ± 1.48 | 8.31 ± 1.61 | 7.98 ± 1.55 |
| mmol/L | | | |

Results are expressed as mean ± SD (n = 6).
Units of mg/kg represent the active drug composition.
Abbreviations:
RBC, red blood cells;
HGB, hemoglobin;
HCT, hematocrit;
MCV, mean cell volume;
MCH, mean cell hemoglobin;
MCH, mean cell hemoglobin concentration;
PLT, platelets;
WBC, white blood cells;
Neut, neutrophils;
Lymph, lymphocytes;
Mono, monocytes;
Eo, eosinophils;
Baso, basophils;
ALP, alkaline phosphatase;
AST, aspartate aminotransferase;
ALT, alanine aminotransferase;
BUN, blood urea nitrogen.

On the basis of these results, it was concluded that the paclitaxel-encapsulated targeted lipid-polymeric nanoparticle formulation was well tolerated in mice, with a ≧3.5-fold improvement in tolerability versus paclitaxel. Considering that the intended doses for subsequent anti-proliferative studies (≦1 mg/kg paclitaxel) were well below tolerability limits for i.v. administration, the MTD studies indicated the feasibility of efficacy trials using this biocompatible and biodegradable formulation.

### Example 6

### Rat Carotid Injury Model Efficacy Studies

Materials and Methods

Rat Carotid Injury Model and Neointimal Proliferation Studies

35 male Sprague-Dawley rats weighing approximately 450 g were obtained from Charles River Laboratories. Animals were given aspirin (20 mg/kg) by oral gavage and heparin (250 IU/kg) by i.v. injection immediately before surgery. Animals were anesthetized intramuscularly (i.m.) with ketamine (60 mg/kg)/xylazine (10 mg/kg) and buprenorphine as an analgesic. Rat carotid injury was performed as described by Cohen-Sela E, et al. J Control Release. 2006; 113(1):23-30; Tulis D A. Methods Mol. Med. 2007; 139:1-30. The left common carotid artery was denuded of endothelium by three intraluminal passages of a Fogarty arterial embolectomy 2F balloon catheter (Model 120602F, Edwards Lifesciences) in a rotating fashion. Lidocaine hydrochloride was gently swabbed onto the exposed carotids. The arteriotomy site was ligated and treated with bactericide gel. Animals were given additional buprenorphine and allowed to recover on 37° C. heated pads for 1 h.

Immediately after ligation of the arteriotomy site, samples were i.v. injected into the tail vein. A second i.v. dose was given on Day 5. At two weeks, animals were sacrificed by CO₂ inhalation. H&E and Movat Pentachrome stained sections of major organs were examined and both carotids were harvested for computerized morphometric analysis.

Histological Morphometric Analysis

Tissues were fixed in 4/100 paraformaldehyde, 4/100 sucrose in saline (9 g NaCl in 1 L H₂O) overnight at 4° C. Tissues were paraffin-embedded and sectioned to give nine representative arterial cross-sections across the length of the artery and H&E stained (AML Laboratories, Rosedale, Md.).

Images were obtained with a Zeiss microscope using the bright field setting at 5× objective. Using the NIH Image computerized morphometric analysis software (http://rsb.info.nih.gov/ij/), a blinded investigator analyzed the degree of neointimal thickening which is expressed as the ratio between the neointimal and medial areas (N/M), From nine individual images sampled, the cross-section with the highest N/M ratio gives the site of greatest luminal narrowing and this value is assigned to the artery. Determination of N/M ratios of carotid samples (Cohen-Sela E, et al. J Control Release. 2006; 113(1):23-30): Using an injury-only group carotid artery cross-section as an example, three rings were made to surround the (i) tunica media (at the external elastic lamina, EEL), (ii) tunica intima (at the internal elastic lamina, IEL) and (iii) lumen. Next, the areas denoted by these three rings were noted. The medial area (M) is calculated from the area bordered by the EEL and the IEL, i.e. area (i) subtract area (ii). The neointimal area (N) is calculated from the area bordered by the IEL and the lumen, i.e. area (ii) subtract area (iii). From there, N/M ratios can be derived (no units).

Immunohistochemistry Studies

Immunohistochemistry of α-SMC actin (SMA) was carried out using standard protocols (Cohen-Sela E, Rosenzweig O, et al. J Control Release. 2006; 113:23-30) with antibodies raised against α-SMA (DAKO, Carpinteria, Calif.). Paraffin-embedded cryosectioned slides were deparaffinized and rehydrated, and then washed in a 1/100 volume ratio of H₂O₂ in methanol for 10 min to quench endogenous peroxidase activity. Non-specific antibody binding was blocked by incubating the slides with 1/10 horse serum in PBS for 20 min. Primary antibodies raised against rat α-SMC (DAKO, Carpinteria, Calif.) were applied for 1 h at 37° C. to detect SMC and neointima. Sections were washed with PBS and incubated with biotinylated secondary antibodies (horse anti-mouse IgG, Vector Laboratory, Burlingame, Calif.) followed by avidin-biotin-peroxidase complexes (ABC Elite kit, Vector Laboratory) for 30 min each. Color development was achieved by a 5 min exposure to the substrate for the HRP reaction, 3,3′-diaminobenzidine tetrahydrochloride. Slides were lightly counterstained with Gill No. 3 hematoxylin to visualize overall tissue morphology. Positive staining was evaluated using a Zeiss microscope under a bright field setting at the 5× objective.

Statistical Analysis

Comparisons of histological findings between control and treatment groups were made using one-way ANOVA with Tukey post-hoc tests. Differences were termed statistically significant at P<0.05. Origin 8 software (OriginLab Corporation, Northampton, Mass.) was utilized to perform this analysis.

Results

A rat carotid balloon-injury model was used to investigate the ability of the targeted lipid-polymeric nanoparticles to inhibit cellular proliferation after arterial injury. The injury from repeated inflation and withdrawal of the catheter induces endothelial cell loss and intimal damage. To prevent post-procedural acute thrombosis, rats were given a single oral aspirin and i.v. heparin bolus. Representative H&E stained carotid artery cross-sections taken on Day 0 of the surgery showed the loss of an endothelial monolayer from arterial balloon-injury when compared to non-injured arteries. At two weeks, Movat Pentachrome stained cross-sections of balloon-injured left carotids showed extensive neointimal proliferation and luminal narrowing compared to healthy right carotids.

Three treatment groups of paclitaxel, NP, and targeted lipid-polymeric nanoparticles were compared against sham injury-only groups. Paclitaxel samples were given as an i.v. bolus injection at either 0.3 mg/kg or 1 mg/kg, with five animals per treatment dose. Repeat dosing is non-invasive and may be beneficial in preventing neointimal proliferation (Kolodgie F D, et al. Circulation. 2002; 106:1195-1198). Paclitaxel levels (tracked by ¹⁴C-paclitaxel) in circulation could not be further detected after 24 h (FIG. 7D), while targeted lipid-polymeric nanoparticle levels (tracked by ³H-PLGA) were detected as late as 120 h (FIG. 7D). Day 5 was chosen for the repeat dose to avoid stacked drug dosing, as it was hypothesized that circulatory and arterial tissue levels of paclitaxel would be clinically insignificant by 120 h. Therefore, all the treatment groups were given two doses on Day 0 and Day 5, midway to the conclusion of the study. The surgical procedure itself and sample dosing did not cause mortality or any apparent morbidity. The mean weights of animals were measured daily; Table 3 shows the average weights of each treatment group at Day 0 (pre-procedure), Day 7 and Day 14. During the study, all animals including sham-operated animals lost up to 0.05 of their original mean body weight at Day 7 and gained 0.1 of their original mean body weight by Day 14 (all vs. Day 0).

TABLE 3
Time course of body weight in Sprague-Dawley rat carotid model (g).

| | | | % ΔBW | | % ΔBW |
|---|---|---|---|---|---|
| | Day 0 | Day 7 | Day 7 − Day 0 | Day 14 | Day 14 − Day 0 |
| Sham, injury only | 458 ± 8 | 447 ± 15 | 1.02 ± 0.03 | 493 ± 15 | 1.13 ± 0.03 |
| paclitaxel, mg/kg | | | | | |
| 0.3 | 460 ± 5 | 452 ± 8 | 0.97 ± 0.02 | 497 ± 11 | 1.07 ± 0.02† |
| 1 | 459 ± 4 | 456 ± 10 | 0.96 ± 0.02 | 488 ± 11 | 1.03 ± 0.02 |
| NP, mg/kg | | | | | |
| 0.3 | 457 ± 6 | 448 ± 7 | 0.98 ± 0.00 | 507 ± 13 | 1.13 ± 0.02† |
| 1 | 450 ± 4 | 433 ± 4 | 0.96 ± 0.01 | 487 ± 6 | 1.08 ± 0.01† |
| Pep-NP, mg/kg | | | | | |
| 0.3 | 455 ± 2 | 433 ± 7 | 0.96 ± 0.02* | 486 ± 5 | 1.08 ± 0.01† |
| 1 | 458 ± 5 | 452 ± 10 | 0.98 ± 0.02 | 495 ± 10 | 1.08 ± 0.02† |

Results are expressed as mean ± SEM (n = 5).
Sham, injury-only groups received balloon-angioplasty and saline.
Units of mg/kg represent the active drug composition.
Changes in body weight are expressed as percentage delta body weight (% ΔBW) of Day 0, mean ± SEM.
Significant differences present horizontally: *P < 0.05 Day 7 vs. Day 0; †P < 0.05 Day 14 vs. Day 0.
There were no significant differences present vertically on Day 0, 7, and 14 against sham, injury only groups.

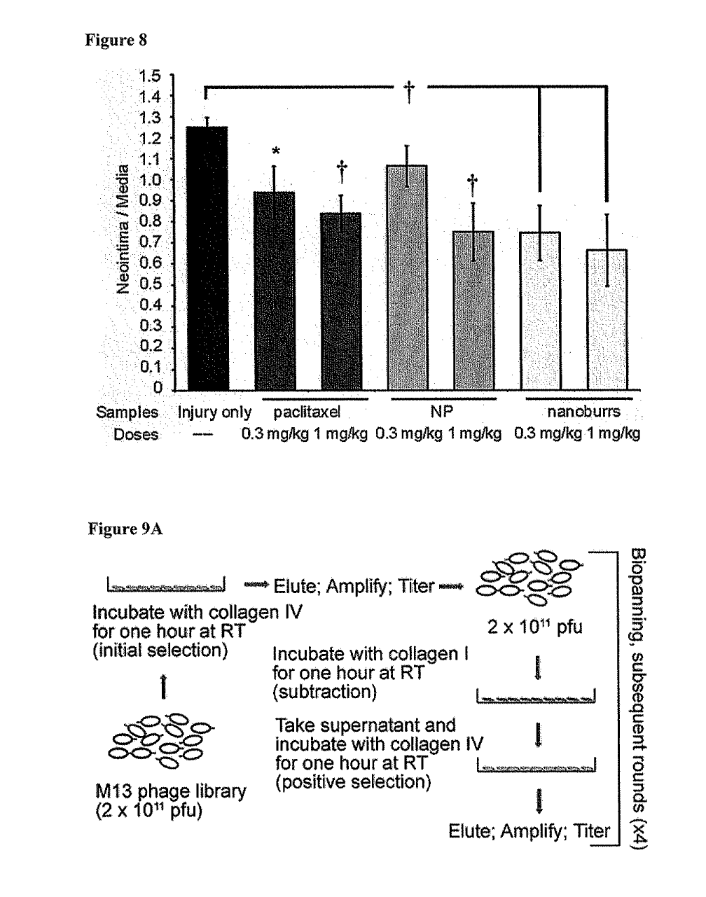

At two weeks, animals were sacrificed and both carotids were harvested for morphometric quantification using ImageJ software (NIH). The degree of neointimal thickening was denoted as a unitless ratio of neointima-to-media (N/M) area. N/M measurements taken from the site of greatest luminal narrowing per artery (FIG. 8) showed that sham, injury-only groups had N/M scores of N/Mₛₕₐₘ=1.249±0.046 versus 1 mg/kg treatment groups of N/M_{paclitaxel}=0.837±0.087, N/M_{NP}=0.749±0.136 and N/M_{targeted lipid-polymeric nanopeptide}=0.662±0.169 (all P<0.01 vs. injury-only group, mean±SEM, n=5). With doses lowered three-fold to 0.3 mg/kg, average NIM ratios per treatment group were N/M_{paclitaxel}=0.937±0.126 (P<0.05 mean±SEM, n=5), N/M_{NP}=1.063±0.097 (P>0.05 mean±SEM, n=5) and N/M_{targeted lipid-polymeric nanoparticle}=0.744±0.129 (P<0.01 mean±SEM, n=5) (FIG. 8). The anti-restenotic efficacy observed with low therapeutic doses suggests the improved potency of paclitaxel treatment by localization of the targeted lipid-polymeric nanoparticles to the site of injury.

Representative images taken with H&E staining show qualitative differences in the thickness of the neointima (N) in relation to the media (M) when compared to injury-only saline groups (FIG. 8). The neointimal proliferation seen here establishes an unambiguous dose-response relationship when different doses of paclitaxel are given, and also the contribution of targeting in the targeted lipid-polymeric nanoparticle treatment groups, in particular the 1 mg/kg dose targeted lipid-polymeric nanoparticle group.

In representative α-smooth muscle cell actin (α-SMA) immunostained cross-sections, no specific staining was observed in the non-angioplastied right artery, whereas a high intensity of SMA positive cells and neointima was observed in the non-treated angioplastied left artery and in the 0.3 mg/kg dose groups. Overall, the targeted lipid-polymeric nanoparticle treated groups showed improved lumen patency and reduction in α-SMA staining.

Examination of H&E stained sections of vital organs gave only incidental findings and no signs of toxicity.

Modifications and variations of the present invention will be obvious to those skilled in the art and are intended to come within the scope of the scope of the appended claims.

We claim:

1. An isolated peptide having the amino acid sequence SEQ ID NO:6, wherein the peptide binds to endothelial basement membrane protein exposed within the leaky junctions.

2. The isolated peptide of claim 1 selected from the group consisting of SEQ ID NO:1, SEQ ID NO:2, SEQ ID NO:3, SEQ ID NO:4, and SEQ ID NO:5.

3. The isolated peptide of claim 1 having a length of 2 to 20 amino acids.

4. The isolated peptide of claim 1 conjugated to a therapeutic, prophylactic, nutraceutical, diagnostic or barrier molecule.

5. The isolated peptide of claim 1 in a fusion protein.

6. The isolated peptide of claim 1 bound to a substrate or particle.

7. A composition for selectively binding to endothelial basement membrane comprising the peptide of claim 1 bound to a substrate or therapeutic, nutritional, diagnostic or prophylactic agent.

8. The composition of claim 7, wherein the substrate is selected from the group consisting of nanoparticles, microparticles, medical devices, foams, sponges, film, wound healing materials, and tissue engineering materials.

9. The composition of claim 8, wherein the peptide is bound to a nanoparticle or microparticle.

10. The composition of claim 8, wherein the medical device is a stent or catheter.

11. The composition of claim 7, wherein the therapeutic, nutritional, diagnostic or prophylactic agent is a chemotherapeutic agent.

12. The composition of claim 7, wherein therapeutic, nutritional, diagnostic or prophylactic agent modifies blood vessel growth, repair or proliferation.

13. A method of treating a disorder or disease characterized by leaky junctions, altered basement membrane permeability, or denuded epithelia or endothelia comprising administering a composition comprising a therapeutic, prophylactic or diagnostic agent conjugated to a targeting ligand that specifically or selectively binds to an endothelial basement membrane protein exposed within the leaky junctions.

14. The method of claim 13, wherein the endothelial basement membrane protein is selected from the group consisting of type I, II, III, or IV collagen fibers; perlecan; laminins; integrins; entactins, and dystroglycans.

15. The method of claim 14, wherein the endothelial basement membrane protein is Collagen IV or laminin.

16. The method of claim 13, wherein the disease or disorder is selected from the group consisting of tumors, restenosis, transplantation, ophthalmologic involving the vasculature, sepsis, and prematurity in infants.

17. The method of claim 13, wherein the pharmaceutical agent is a barrier molecule which decreases flow through the leaky junctions.

18. The method of claim 13, wherein the targeting ligand is bound to a micro or nanoparticle.

19. The method of claim 13, wherein the targeting ligand comprises the isolated peptide of claim 1 or the composition of claim 7.

20. A method for diagnosing leaky junctions, altered basement membrane permeability, or denuded epithelia or endothelia, or a disease or disorder thereof, comprising detecting a reaction between the isolated peptide of claim 1 or the composition of claim 7 and an endothelial basement membrane protein exposed during the disorder or disease and not in normal endothelium.

21. A method for identifying agents for treating a disorder or disease characterized by leaky junctions, altered basement membrane peimeability, or denuded epithelia or endothelia, comprising
contacting an endothelium or epithelium with the isolated peptide of claim 1 or the composition of claim 7 and a candidate agent, and
detecting a reaction between the peptide or composition and the endothelial basement membrane protein,
wherein a detectable decrease in peptide or composition binding to the endothelial basement membrane protein compared to a control is an indication that the candidate agent is useful for treating a disorder or disease characterized by leaky junctions, altered basement membrane permeability, or denuded epithelia or endothelia.

22. A method of treating a subject in need thereof comprising administering to subject the composition of claim 7.

23. The method of claim 22, wherein the subject has cancer or an angiogenic mediated disease or disorder.

24. The method of claim 22, wherein the subject has undergone vascular repair or disruption or transplantation.

25. The method of claim 22, wherein the subject has a vascular disorder or disease of the eye.

26. A composition for use in the method of claim 13 comprising a pharmaceutical agent conjugated to a targeting ligand that specifically or selectively binds to an endothelial basement membrane protein exposed within the leaky junctions.
